(19) United States
(12) Patent Application Publication
(10) Pub. No.: US 2024/0058519 A1
(43) Pub. Date: Feb. 22, 2024

(54) DISPOSABLE CIRCUIT FOR EXTRACORPOREAL TREATMENT OF BLOOD, APPARATUS FOR EXTRACORPOREAL TREATMENT OF BLOOD AND ASSOCIATED METHOD

(71) Applicant: GAMBRO LUNDIA AB, Lund (SE)

(72) Inventor: Dominique Pouchoulin, Tramoyes (FR)

(21) Appl. No.: 18/270,122

(22) PCT Filed: Oct. 11, 2021
(86) PCT No.: PCT/EP2021/078070

§ 371 Date: Jun. 28, 2023

(30) Foreign Application Priority Data
Dec. 29, 2020 (EP) 20306706.1

(51) Int. Cl. A61M 1/36; A61M 39/10

(52) U.S. Cl. CPC A61M 1/3644; A61M 39/10

(57) ABSTRACT

A disposable circuit for extracorporeal blood treatment comprising a filtration unit (2) and a blood circuit (6, 7) with a blood withdrawal line (6) and a blood return line (7), said blood withdrawal line (6) and said blood return line (7) being designed to be connected to a patient cardiovascular system, wherein the blood withdrawal line (6) and the blood return line (7) are provided with an arterial connector (40) and a venous connector (41) detachably connected to a vascular access device of a patient; at least one fluid line (15, 21, 25, 42; 42a) is connected to the blood circuit (6, 7) and the disposable circuit further comprises a first auxiliary connector (51, 53, 55, 57, 59) and a second auxiliary connector (50, 52, 54, 56, 58) arranged either on the blood circuit (6, 7) or on the fluid line (15, 21, 25, 42; 42a), and configured to be removably connected with the arterial connector (40) and the venous connector (41) in a recirculation configuration so as to define a closed circuit allowing fluid recirculation.

### FIELD OF THE INVENTION

The present disclosure relates to a disposable circuit for extracorporeal treatment of blood configurable for fluid recirculation, to an apparatus for extracorporeal treatment of blood, a method of blood flow recirculation in the disposable circuit and an associated computer program product.

The disposable circuit is particularly suitable for temporary disconnection of a patient from the apparatus for extracorporeal treatment of blood and subsequent reconnection to the same disposable circuit for treatment prosecution.

### BACKGROUND ART

Extracorporeal blood treatment involves removing blood from a patient, treating the blood externally to the patient, and returning the treated blood to the patient. Extracorporeal blood treatment is typically used to extract undesirable matter or molecules from the patient's blood and/or add desirable matter or molecules to the blood.

Extracorporeal blood treatment is used with patients unable to effectively remove matter from their blood, such as when a patient has suffered temporary or permanent kidney failure. These patients and other patients may undergo extracorporeal blood treatment to add or remove matter to their blood, to maintain an acid/base balance or to remove excess body fluids, for example.

Extracorporeal blood treatment is typically accomplished by removing the blood from the patient in e.g. a continuous flow, introducing the blood into a primary chamber, also referred to as blood chamber, of a filtration unit (such as a dialyzer or a hemofilter) where the blood is allowed to flow past a semipermeable membrane. The semipermeable membrane selectively allows matter in the blood to cross the membrane from the primary chamber into a secondary chamber and also selectively allows matter in the secondary chamber to cross the membrane into the blood in the primary chamber, depending on the type of treatment.

A number of different types of extracorporeal blood treatments may be performed. In an ultrafiltration (UF) treatment, undesirable matter is removed from the blood by convection across the membrane into the secondary chamber. In a hemofiltration (HF) treatment, the blood flows past the semipermeable membrane as in UF and desirable matter is added to the blood, typically by dispensing a fluid into the blood either before and/or after it passes through the filtration unit and before it is returned to the patient. In a hemodialysis (HD) treatment, a secondary fluid containing desirable matter is introduced into the secondary chamber of the filtration unit. Undesirable matter from the blood crosses the semipermeable membrane into the secondary fluid and desirable matter from the secondary fluid may cross the membrane into the blood. In a hemodiafiltration (HDF) treatment, blood and secondary fluid exchange matter as in HD, and, in addition, matter is added to the blood, typically by dispensing a fluid into the treated blood before its return to the patient as in HF.

Specific blood treatment apparatuses have been developed for the treatment of acute patients mainly because:
in acute patients, the total treatment time is a priori unknown and, as such, it may not be used as setup parameter as it is not known how long kidney insufficiency will be present; indeed, acute patients often need relatively long treatment sessions, typically lasting several days, before recovering from kidney failure;
intensive care apparatus may rather be designed to request a plurality of flow rate information as setup parameters;
acute patients need a very precisely controlled fluid removal; moreover, infusion of drugs or of fluids in general shall also be very accurately controlled;
acute patients are also very weak; thus, in the course of the treatment of acute patients it has been regarded as relevant to make sure that a continuous and gentle therapy is delivered to the patient during treatment with a very accurate control on the flow rate in each of the lines connected with the bloodlines;
furthermore, apparatus often need to be operated in emergency situations, thus apparatus for acute treatment shall be characterized by very easy to set up procedures.

Some treatments for patients may involve very long procedures wherein blood is treated for several days or even weeks; this is the case of continuous renal replacement therapies CRRT wherein the blood treatment apparatuses may be operated continuously for some days. Irrespective of whether the treatment is for continuous renal replacement, or for acute or chronic patients, there may be cases wherein the patient may need to be disconnected from the apparatus when the treatment is still not yet completed. This is the case for instance when the patient shall be subjected to medical exams to be performed outside the renal unit room or when an intervention on the vascular access shall be suddenly performed. Disconnecting and reconnecting the blood treatment apparatus to the blood circuit lines requires disconnecting these lines from the patient, in particular from a venous connector and an arterial connector arranged on the ends of the blood circuit apparatus (for example the patient is disconnected from respective venous and arterial needles inserted into the fistula—chronic treatments—or from a catheter fixed to the patient-acute treatments).

Such temporary disconnection is critical at least for the following reasons. Disconnection and reconnection of blood circuit of the blood treatment apparatuses shall be performed in sterile conditions, and this requires a qualified operator who may not interact with the control unit of the apparatus, which is typically not sterile. Stagnant blood in the blood circuit of the apparatus may coagulate; therefore, the blood treatment apparatus shall be left non-operative for very short times to avoid harming the patient safety. On the other hand, complete substitution of disposable circuits during temporary interruption of the therapy should be avoided at least for an economic reason; in this latter case, substitution of disposable circuits would imply significantly higher costs for each single treatment.

Document U.S. Pat. No. 9,713,670 discloses a method for temporarily interrupting an extracorporeal treatment of blood of a patient by use of a blood treatment apparatus. The method comprises activating a control device configured to bring the blood treatment apparatus into a state in which the blood treatment session of the patient can be interrupted with the intention to continue the blood treatment session. The ends of the blood lines are disconnected from the patient and connected through an ‘Y’ connector to the same saline solution bag. The fluid is then recirculated inside the blood circuit.

Document US2009/221949 describes a device for circulatory isolation and treatment of a part of a patient body. A pump is aspirating liquid from a venous reservoir for sending the liquid to a heater and oxygenator unit. An outlet of the oxygenator unit is connected to a first arterial Y-connector. The mainstream outlet of the first arterial Y-connector is connected via an arterial trunk conduit to the patient's arterial catheter. On the venous side, blood or liquid coming from the patient via a venous catheter passes a venous front conduit and a venous Y-connector. A shunt inlet of the venous Y-connector is connected to a shunting outlet of the first arterial Y-connector by means of a shunt conduit. The mainstream outlet is connected to the venous reservoir with an integrated filter. A third port of connector may send liquid to a collecting bag 159.

Document U.S. Pat. No. 6,913,588 shows an extracorporeal circuit connected in line with an existing hemofiltration system. The hemofiltration system emerges from a patient venous connection in a conduit provided with a blood pump. An anticoagulant and replacement fluids are infused into the conduit with a pump to prevent blood coagulation and replace blood volume lost as a waste product. Blood enters a hemofilter where ultrafiltrate is separated from the blood. After filtration, blood leaves the hemofilter along a bloodline, through a shunt and a conduit, returning to a patient arterial system. Ultrafiltrate leaves the hemofilter at an ultrafiltrate port through the ultrafiltrate conduit.

Document U.S. Pat. No. 5,630,946 discloses a method for removing leukocytes from the blood in an extracorporeal circuit by passing a leukocyte containing blood from a patient into a container, passing a portion of biological fluid from the container through a recirculation loop including a filter assembly for removing leukocytes and moving the portion of leukocyte-depleted blood into the container; additional blood with leukocyte is transferred from a patient into the container so that the additional blood is mixed with leukocyte depleted blood in the container to provide a mixture and then a portion of the mixture is sent to the patient while passing another portion of the mixture through the recirculation loop.

It is an object of embodiments of the present disclosure to provide a disposable circuit for extracorporeal treatment of blood particularly suitable for intensive care, but also for chronic care applications which helps a rapid and safe temporary disconnection of the patient from the apparatus during the execution of a blood treatment.

Furthermore, it is an object of embodiments of the disclosure a disposable circuit for extracorporeal treatment of blood, which is economically convenient to use.

It is a further object of embodiments of the disclosure a disposable circuit for extracorporeal treatment of blood, which prevents errors of fluid management for the fluids still resting in the apparatus while the patient is temporarily disconnected therefrom.

It is a further object of embodiments of the disclosure a disposable circuit for extracorporeal treatment of blood which allows a safe reconnection of the patient to the apparatus, and a safe reintroduction of the fluid and/or blood still present in the apparatus into the circulatory apparatus of the patient when this latter is reconnected to the treatment machine.

It is a further object of embodiments of the disclosure to provide a disposable circuit that, when the patient is disconnected from the apparatus, is configurable in a configuration which prevents stagnation and/or reduces the risk of coagulation of the blood still present therein.

### SUMMARY

At least one of the above objects is substantially reached by a disposable circuit according to the following aspects.

The disposable circuit is disclosed in accordance to the following aspects, which may be combined together in any combination when possible. Thus, dependences of the aspects shall not be intended to be strictly limiting.

According to a first aspect, is herewith disclosed a disposable circuit for extracorporeal treatment of blood comprising:
a filtration unit (2) in particular having a primary chamber (3) and a secondary chamber (4) separated by a semi-permeable membrane (5);
a blood circuit (6, 7) comprising a blood withdrawal line (6) having a first end connected to an inlet of the filtration unit (2), in particular of the primary chamber (3), and a blood return line (7) having a first end connected to an outlet of the filtration unit (2), in particular of the primary chamber (3), said blood withdrawal line (6) and said blood return line (7) being designed to be connected to a patient cardiovascular system, wherein the blood withdrawal line (6) has a second end provided with an arterial connector (40) and the blood return line (7) has a second end provided with a venous connector (41), said arterial connector (40) and said venous connector (41) being designed to be detachably connected to a vascular access device of a patient; and wherein said blood circuit is configured to be interfaced with a blood pump (11) for controlling the flow in the blood circuit (6, 7);
optionally an effluent fluid line (13) connected to an outlet of the secondary chamber (4);
at least one fluid line (15, 21, 25, 42; 42a) connected to the blood circuit (6, 7), the disposable circuit further comprising at least one first auxiliary connector (51, 53, 55, 57, 59) arranged either on the blood circuit (6, 7) or on said at least one fluid line (15, 21, 25, 42; 42a), said at least one first auxiliary connector (51, 53, 55, 57, 59) being configured to be removably connected with at least one connector between the arterial connector (40) and the venous connector (41) in a recirculation configuration so as to define a closed circuit allowing fluid recirculation in the blood circuit; said disposable circuit optionally further comprising a second auxiliary connector (50, 52, 54, 56, 58) arranged either on the blood circuit or on said at least one fluid line (15, 21, 25, 42; 42a), and wherein the first auxiliary connector (51, 53, 55, 57, 59) and the second auxiliary connector (50, 52, 54, 56, 58) are configured to be removably connected one with the arterial connector (40) and the other with the venous connector (41) so as to define said closed circuit.

This first aspect is for example directed (but not limited) to an apparatus for performing dialysis treatments, such as HF and HDF and treatments including regional anticoagulation, or hemoperfusion treatments with fluid infusion. Further, this first aspect may also be related to a configuration for ECCO2R with infusion, namely use of an oxygenator placed on the blood line to remove excess CO₂ from blood.

According to another independent aspect a disposable circuit for extracorporeal treatment of blood comprises:
a filtration unit (2);
a blood circuit (6, 7) comprising a blood withdrawal line (6) having a first end connected to an inlet of the filtration unit (2), and a blood return line (7) having a first end connected to an outlet of the filtration unit (2), said blood withdrawal line (6) and said blood return line (7) being designed to be connected to a patient cardiovascular system,
wherein the blood withdrawal line (6) has a second end provided with an arterial connector (40) and the blood return line (7) has a second end provided with a venous connector (41), said arterial connector (40) and said venous connector (41) being designed to be detachably connected to a vascular access device of a patient; and wherein said blood circuit is configured to be interfaced with a blood pump (11) for controlling the flow in the blood circuit (6, 7);
at least one first auxiliary connector (51, 53, 55, 57, 59) arranged on the blood circuit (6, 7), said at least one first auxiliary connector (51, 53, 55, 57, 59) being configured to be removably connected with at least one connector between the arterial connector (40) and the venous connector (41) in a recirculation configuration so as to define a closed circuit allowing fluid recirculation in the blood circuit;
said disposable circuit optionally further comprising a second auxiliary connector (50, 52, 54, 56, 58) arranged on the blood circuit, and wherein the first auxiliary connector (51, 53, 55, 57, 59) and the second auxiliary connector (50, 52, 54, 56, 58) are configured to be removably connected one with the arterial connector (40) and the other with the venous connector (41) so as to define said closed circuit.

This additional aspect is for example directed (but not limited) to an apparatus for performing dialysis treatments, such as HD, with no infusions into the blood circuit or hemoperfusion treatments with no infusions. Further, this further aspect may also be related to a configuration for ECCO2R without infusions, namely use of an oxygenator placed on the blood line to remove excess CO₂ from blood.

According to a further aspect depending on the previous aspect, the disposable circuit is reversibly configurable between a treatment configuration, wherein the arterial connector (40) and the venous connector (41) are removably connected to the vascular access device of a patient and blood may be withdrawn from the patient through the blood withdrawal line (6), circulated through the filtration unit (2) and returned to the patient through the blood return line (7), and a recirculation configuration, wherein one of the arterial connector (40) and venous connector (41) is removably connected to one of the first auxiliary connector (51, 53, 55, 57, 59) and the second auxiliary connector (50, 52, 54, 56, 58), optionally the other one of the arterial connector (40) and venous connector (41) is removably connected to the other one of the first auxiliary connector (51, 53, 55, 57, 59) and the second auxiliary connector (50, 52, 54, 56, 58), wherein in the recirculation configuration a fluid contained in the blood circuit (6, 7) may be recirculated inside the disposable circuit.

According to a 2nd aspect depending on any one of the previous aspects, the first auxiliary connector (51, 53, 55, 57, 59), optionally both the first auxiliary connector (51, 53, 55, 57, 59) and the second auxiliary connector (50, 52, 54, 56, 58), is/are provided with at least one disconnectable port (70; 70a) configured to be connected to corresponding a disconnectable port (72) of either the venous connector (41) or the arterial connector (40).

A disconnectable port refers to an inlet or an outlet of a connector allowing for a removable connection with a corresponding disconnectable port of another connector. A Luer lock design is an example of removable connection, wherein one disconnectable port of the first connector may be a tapered cone configured to slip onto its mating tapered cavity of the second connector; the other disconnectable port on the second connector is the mating tapered cavity with integrated locking threads.

According to a 3rd aspect depending on aspect 2, at least one connector between the first auxiliary connector (51, 53, 55, 57, 59) and the second auxiliary connector (50, 52, 54, 56, 58), optionally both the first auxiliary connector (51, 53, 55, 57, 59) and the second auxiliary connector (50, 52, 54, 56, 58), is/are three ports connector/s, provided with a disconnectable port (70) configured to be connected to a disconnectable port (72) the venous connector (41) or the arterial connector (40).

According to a 4th aspect depending on one or more of aspects 1-2, the first auxiliary connector (51, 53, 55, 57, 59) is a four port connector provided with a first disconnectable port (70) and a second disconnectable port (70a) each of which is configured to be connected to a respective disconnectable port (72) of the venous connector (41) or the arterial connector (40).

According to a 5th aspect depending on any of aspects 2 or 3 or 4, at least one connector between the first auxiliary connector (51, 53, 55, 57, 59) and the second auxiliary connector (50, 52, 54, 56, 58), optionally both said first auxiliary connector (51, 53, 55, 57, 59) and the second auxiliary connector (50, 52, 54, 56, 58), is/are provided with:
a first non-disconnectable service port (71) fixed to a tube portion, optionally a first and a second non-disconnectable service port (71) fixed to respective tube portions;
said at least one disconnectable port (70).

According to a 6th depending on aspect 4, the first auxiliary connector (51, 53, 55, 57, 59) is provided with a first and a second non-disconnectable service port (71) fixed to a tube portion, said first disconnectable port (70) and said second disconnectable port (70a), wherein said first and second non-disconnectable service ports (71) fixed to respective tube portions are configured to be non-removably connected to respective portions of the fluid line (15, 21, 25, 42; 42a) at which the first auxiliary connector is fixed.

According to a 7th aspect depending on one or more of aspects 1-2, at least one connector between the first auxiliary connector (51, 53, 55, 57, 59) and the second auxiliary connector (50, 52, 54, 56, 58) is an end connector provided with said disconnectable port (70) configured to detachably connect with the arterial connector (40) or with the venous connector (41).

According to an 8th aspect depending on aspects 2-7, at least the first auxiliary connector (51, 53, 55, 57, 59) or the second auxiliary connector (50, 52, 54, 56, 58) is a “Y”-shaped or “T”-shaped connector, and/or is a Luer-type connector and/or is a self-sealing connector with at least its first disconnectable port (70) being configured to automatically seal, preventing fluid leakage, upon disconnection from another connector and to automatically open, allowing fluid passage, upon connected to the venous connector (41) or the arterial connector (40).

According to a 9th aspect depending on one or more of the preceding aspects:
said at least one disconnectable port (70; 70a) of the at least one first auxiliary connector (51, 53, 55, 57, 59) and said at least one disconnectable port (72) of the arterial connector (40) or of the venous connector (41) may be either a first-male-type or a second-female-type, said first-male-type being configured to at least partially introduce into the second-female-type;
both the arterial connector (40) and the venous connector (41) are connectors provided with a disconnectable port (72) of the first type, or alternatively,
both the arterial connector (40) and the venous connector (41) are provided with a disconnectable port (72) of the second type.

According to a 10th aspect depending on said aspect 9:
the first auxiliary connector (51, 53, 55, 57, 59) and the second auxiliary connector (50, 52, 54, 56, 58) have the at least one detachable port (70) both of a male type or both of a female type.

According to an 11th aspect depending on one or more of the preceding aspects, said one or more fluid lines (15, 21, 25; 42; 42a) are directly connected to the blood circuit, optionally through the interposition of a non-disconnectable connector, preferably a three-way integral connector.

According to a 12th aspect depending on one or more of the preceding aspects, said one or more fluid lines (15, 21, 25; 42; 42a) comprise one or more of the following lines:
a post-blood pump pre-dilution infusion line (15), directly connected to the blood withdrawal line (6);
a pre-blood pump pre-dilution infusion line (21), directly connected to the blood withdrawal line (6);
a post-dilution fluid line (25), directly connected to the blood return line (7).

According to a 13th aspect depending on one or more of the previous aspects, the first auxiliary connector (51, 53, 55, 57, 59) is arranged on the blood circuit (6, 7).

According to a 14th aspect depending on one or more of the previous aspects, the first auxiliary connector (51, 53, 55) is arranged on the blood withdrawal line (6).

According to a 15th aspect depending on one or more of the previous aspects 1-13, the first auxiliary connector (57, 59) is arranged on the blood return line (7).

According to a 16th aspect depending on one or more of the previous aspects 1-12, the first auxiliary connector (51) is arranged on the post-blood pump pre-dilution infusion line (15).

According to a 17th aspect depending on one or more of the previous aspects 1-12, the first auxiliary connector (53) is arranged on the pre-blood pump pre-dilution infusion line (21).

According to an 18th aspect depending on one or more of the previous aspects 1-12, the first auxiliary connector (57) is arranged on the post-dilution fluid line (25).

According to a 19th aspect depending on one or more of the previous aspects, the second auxiliary connector (50, 52, 54, 56, 58) is arranged on the blood circuit (6, 7).

According to a 20th aspect depending on one or more of the previous aspects, wherein the second auxiliary connector (50, 52, 54, 58) is arranged on the blood withdrawal line (6).

According to a 21st aspect depending on one or more of the previous aspects, the second auxiliary connector (50, 52, 54, 56, 58) is arranged on the blood return line (7).

According to a 22nd aspect depending on one or more of the previous aspects 1-18, the second auxiliary connector (50) is arranged on the post-blood pump pre-dilution infusion line (15).

According to a 23rd aspect depending on one or more of the previous aspects 1-18, the second auxiliary connector (52) is arranged on the pre-blood pump pre-dilution infusion line (21).

According to a 24th aspect depending on one or more of the previous aspects 1-18, the second auxiliary connector (56) is arranged on the post-dilution fluid line (25).

According to a 25th aspect depending on one or more of the previous aspects 1-12, the first auxiliary connector and the second auxiliary connector are arranged in said post-blood pump pre-dilution infusion line (15).

According to a 26th aspect depending on one or more of the previous aspects 1-12, the first auxiliary connector and the second auxiliary connector are arranged in said pre-blood pump pre-dilution infusion line (21).

According to a 27th aspect depending on one or more of the previous aspects 1-12, the first auxiliary connector and the second auxiliary connector are arranged in said post-dilution fluid line (25).

According to a further aspect depending on one or more of the previous aspects 1-12, the first auxiliary connector and the second auxiliary connector are arranged on the blood circuit, for example, the first auxiliary connector on the blood withdrawal line and the second auxiliary connector on the blood return line or, alternatively, both auxiliary connectors are placed on the blood withdrawal line (6).

According to a 28th aspect depending on one or more of the previous aspects, the at least one fluid line (15, 21, 25, 42; 42a) connected to the blood circuit comprises at least one auxiliary line (42, 42a).

According to a 29th aspect depending on one or more of the previous aspects 1-12 and 28, the first auxiliary connector (55) is arranged on the at least one auxiliary line (42, 42a).

According to a 30th aspect depending on one or more of the previous aspects 1-12 and 28 or 29, the second auxiliary connector (54) is arranged on the at least one auxiliary line (42, 42a).

According to a 31st aspect depending on one or more of the previous aspects 28-30, the first auxiliary connector and the second auxiliary connector are installed on a same, single, auxiliary line (42); said auxiliary fluid line (42) being optionally provided with an own fluid pump tract on which a fluid pump operates; optionally the first auxiliary connector and the second auxiliary connector being installed one upstream with respect to the other.

According to a 32nd aspect depending on one or more of the previous aspects 1-31, a blood pump tract, configured to receive blood pump (11) action, is provided with an inlet port and an outlet port, and wherein the withdrawal line (6) comprises a first portion (6a) positioned upstream the inlet port of the blood pump tract and downstream the arterial connector (40) and a second portion positioned downstream the outlet port of the blood pump tract and upstream the inlet of the primary chamber (3).

According to a 33rd aspect depending aspects 17 and 32, the first auxiliary connector (53) of the pre-blood pump pre-dilution infusion line (21) is provided with a port in direct fluid communication with the first portion (6a) of the blood withdrawal line (6).

According to a 34th aspect depending on aspect 33, the pre-blood pump pre-dilution infusion line (21) is configured to allow fluid administration to the blood circuit downstream the arterial connector (40); said pre-blood pump pre-dilution infusion line (21) optionally comprising a container, for example, containing an anticoagulant solution (e.g., a citrate solution), said pre-blood pump pre-dilution infusion line (21) being connected to said container.

According to a 35th aspect depending on aspect 34th, said fluid line comprises:
a first auxiliary line (42), optionally provided with an own fluid pump,
a second auxiliary line (42a), optionally provided with an own fluid pump,
the first auxiliary connector being installed in the first auxiliary line (42), the second auxiliary connector being installed in the second auxiliary line (42a).

According to a 36th aspect, depending on one or more of the previous aspects, the disposable circuit further comprises at least one first pressure drop valve or flow resistor (60) configured for even fluid flow distribution during recirculation, said at least one first pressure drop valve or flow resistor (60) being arranged on the blood circuit or on the at least one fluid line (15, 21, 25, 42; 42a), said at least one first pressure drop valve or flow resistor (60) being configured to even respective fluid flow resistances of two connected branch tube portions during recirculation.

According to a 37th aspect depending on the aspect 36, said at least one first pressure drop valve or flow resistor (60) is arranged in one of the two connected branch tube portions; optionally said two connected branch tube portions comprising at least one portion of the fluid line (15, 21, 25, 42, 42a) and at least one portion of said blood circuit (6, 7).

According to a 38th aspect depending on aspect 36 and on aspect 37, said at least one first pressure drop valve or flow resistor (60) is installed downstream at least one of the first auxiliary connector (51, 53, 55, 57, 59) or second auxiliary connector (50, 52, 54, 56, 58), optionally wherein said at least one first pressure drop valve or flow resistor (60) is installed downstream the first auxiliary connector (51, 53, 55, 57, 59) and downstream the second auxiliary connector (50, 52, 54, 56, 58) and in a portion of fluid line in direct connection with the inlet of the of the primary chamber (3) and, preferably, also in direct connection with the outlet of the blood pump (11).

Downstream is here intended in respect to the infusion fluid direction during substitution fluid pre-infusion during treatment. In particular, the first pressure drop valve or flow resistor (60) is placed on the post-blood pump pre-dilution infusion line (15) downstream the first and second auxiliary connectors (see FIG. 6).

According to a 39th aspect depending on one or more of the previous aspects, at least one of the fluid lines (15, 21, 25, 42; 42a) is configured to be coupled to a flow-regulating actuator, in particular a fluid pump (18, 22, 27) configured to regulate the flow of the fluid in the respective fluid line.

According to a 40th aspect, depending on aspects 12 and 39:
the post-blood pump pre-dilution infusion line (15), connected to the blood withdrawal line (6), has a fluid pump tube section configured to be coupled to at least one infusion pump (18) for regulating the flow of fluid through said pre-dilution fluid line (15);
the post-dilution fluid line (25), connected to the blood return line, has a fluid pump tube section configured to be coupled to at least one infusion pump (27) for regulating the flow through said post-dilution fluid line;
the pre-blood pump pre-dilution infusion line (21), connected to the blood withdrawal line (6), has a fluid pump tube section configured to be coupled to at least one pre-blood infusion pump (22) for regulating the flow through said pre-blood pump infusion line.

According to a 41th aspect, depending on said 40th aspect, at least one connector between the first auxiliary connector (51, 53, 55, 57, 59) and the second auxiliary connector (50, 52, 54, 56, 58) is arranged downstream a fluid pump tube section of the respective infusion fluid line (15, 21, 25), particularly wherein the first auxiliary connector (51, 53, 55, 57, 59) and the second auxiliary connector (50, 52, 54, 56, 58) are arranged downstream the fluid pump (18, 22, 27) of the respective fluid line (15, 21, 25).

According to a 42th aspect, depending on one or more of the previous aspects:
the first auxiliary connector (51, 53, 55, 57, 59) is connectable to the arterial connector (40), in particular through a direct connection between a port of the first auxiliary connector (51, 53, 55, 57, 59) and the arterial connector (40), or through a portion of line connecting a port of the first auxiliary connector (51, 53, 55, 57, 59) and the arterial connector (40); and/or wherein
the second auxiliary connector (50, 52, 54, 56, 58) is connectable to the venous connector (41), in particular through a direct connection between a port of the second auxiliary connector (50, 52, 54, 56, 58) and the arterial connector (40), or through a portion of line connecting a port of the second auxiliary connector (50, 52, 54, 56, 58) and the venous connector (41).

According to a 43th aspect, depending on one or more of the previous aspects, the disposable circuit comprises an air separator, in particular a bubble trap (8), said air separator being placed downstream the outlet of the primary chamber (3), in a portion the blood return line (7) between the outlet of the primary chamber (3) and the venous connector (41), wherein at least one of the fluid lines (15, 21, 25, 42; 42a), preferably all the fluid lines (15, 21, 25, 42; 42a) are connected to the blood circuit (6, 7) upstream or at the air separator, namely between the arterial connector (40) and the air separator.

According to a 44th aspect depending on one or more of the previous aspects, the circuit further comprises a safety clamp (9) active on the blood return line (7) and configured to be controlled by a control unit (10); wherein said at least one fluid line (15, 21, 25, 42; 42a) is placed upstream said safety clamp (9), and optionally wherein all the fluid lines (15, 21, 25, 42; 42a) are placed upstream said safety clamp (9) between said arterial connector (40) and said safety clamp (9).

According to a 45th aspect, depending on aspect 44, at least one connector between said first auxiliary connector (51, 53, 55, 57, 59) and said second auxiliary connector (50, 52, 54, 56, 58) is placed upstream said safety clamp (9), optionally wherein the first auxiliary connector (51, 53, 55, 57, 59) and the second auxiliary connector (50, 52, 54, 56, 58) are both placed upstream said safety clamp (9).

According to a 46th aspect, depending on aspects 42 and 44, at least one connector between said first auxiliary connector (51, 53, 55, 57, 59) and said second auxiliary connector (50, 52, 54, 56, 58) is placed downstream said safety clamp (9), in a portion of the blood return line (7) comprised between the outlet of the primary chamber (3) and the venous connector (41), optionally wherein the first auxiliary connector (51, 53, 55, 57, 59) is placed upstream said safety clamp (9) and the second auxiliary connector (50, 52, 54, 56, 58) is placed downstream said safety clamp (9).

According to a 47th aspect depending on aspect 12, the disposable circuit further comprises at least one infusion fluid container (16, 23, 26) connected with an inlet of said post-blood pump pre-dilution line (15) or with an inlet of said post-dilution fluid line (25) or with an inlet of said pre-blood pump infusion line (21), optionally comprising a first infusion fluid container (16) connected with an inlet of said post-blood pump pre-dilution line (15), a second infusion fluid container (26) connected with an inlet of said post-dilution fluid line (25) and a third infusion fluid container (23) connected with an inlet of said pre-blood pump pre-dilution infusion line (21).

According to a 48th aspect, depending on any of the previous aspects when depending on aspect 43, the disposable circuit interfaces with a bubble sensor (8a), configured for detecting the presence of one or more bubbles in the blood circuit (6, 7); said bubble sensor (8a) being configured to be operatively connected to a control unit (10) and for sending to the control unit (10) a predetermined signal in case of at least one or more bubble is detected in the blood circuit (6, 7).

According to a 49th aspect depending on aspects 44 and 48, said bubble sensor (8a) is associated to the bubble trap (8) or is coupled to a portion of the blood return line (7) between the bubble trap (8) and clamp (9); in particular said bubble sensor (8a) being configured for sending to the control unit (10) a predetermined signal in case of at least one or more bubble is detected, for causing closure of the clamp (9).

According to a further aspect, depending on one or more of the previous aspects, the disposable circuit is a CRRT disposable circuit for use in apparatuses for extracorporeal treatment of blood in the acute field.

According to a further aspect, depending on one or more of the previous aspects, the disposable circuit is a CRRT disposable circuit including disposable dialysis fluid line (19) irremovably connected to the inlet of the secondary chamber (4) and disposable effluent line (13) irremovably connected to the outlet of the secondary chamber (4).

According to a 50th aspect, depending on one or more of the previous aspects, the disposable circuit further comprises a dialysis fluid line (19) connected to the inlet of the secondary chamber (4).

According to a 51st aspect, depending on the 50th aspect, the disposable circuit further comprises a dialysis fluid container (20) provided at an end of said dialysis fluid line (19) and configured to deliver the fluid therein contained to the inlet of said secondary chamber (4);

optionally wherein:
said disposable circuit further comprises a dialysis liquid pump tract placed on the dialysis fluid line (19) and upon which a dialysis liquid pump (17a) operates, to force the supply of fluid from the dialysis liquid container (20) to the inlet of the secondary chamber (4).

According to a 52nd aspect, depending on the aspects 50 or 51, further comprising an effluent fluid container (14) provided at an end of said effluent fluid line (13) and configured to collect the fluid extracted from the outlet of the secondary chamber (4);
said disposable circuit optionally further comprising an effluent liquid pump tract placed on the effluent fluid line (13) and upon which an effluent liquid pump (17), to force the draught of fluid from the outlet of the secondary chamber (4) to the effluent fluid container (14).

According to a 53rd aspect, depending on one or more of the preceding aspects, in said recirculation configuration fluid flows from said arterial connector (40) through said primary chamber (3) to said venous connector (41) or from the second end of the blood withdrawal line (6) to the first end of the blood withdrawal line (6) and from the first end of the blood return line (7) to the second end of the blood return line (7).

According to a 54th aspect depending on one or more of the preceding aspects, said at least one infusion fluid line (15, 21, 25, 42; 42a) comprises a first end connected, in particular directly connected, to the blood circuit (6, 7) and a second end opposite to the first end, the disposable circuit comprising a treatment configuration different from said recirculation configuration, wherein in said treatment configuration extracorporeal blood treatment is performed; in said treatment configuration, in said at least one infusion fluid line (15, 21, 25, 42; 42a) fluid flows from said second end to said first end.

According to a 55th aspect, depending on aspects 12 and 16, in said recirculation configuration the first auxiliary connector (51) arranged on the post-blood pump pre-dilution infusion line (15) is connected to the venous connector (41) and the second auxiliary connector (50) arranged on the post-blood pump pre-dilution infusion line (15) is connected to the arterial connector (40).

According to a 56th aspect, depending on aspects 12 and 16, in said recirculation configuration the first auxiliary connector (51) arranged on the post-blood pump pre-dilution infusion line (15) is connected to the arterial connector (40) and the second auxiliary connector (50) arranged on the post-blood pump pre-dilution infusion line (15) is connected to the venous connector (41).

According to a 57th aspect, depending on aspects 12 and 17, in said recirculation configuration the first auxiliary connector (53) arranged on the pre-blood pump pre-dilution infusion line (21) is connected to the arterial connector (40) and the second auxiliary connector (50) arranged on the pre-blood pump pre-dilution infusion line (21) is connected to the venous connector (41).

According to a 58th aspect, depending on aspects 12 and 17, in said recirculation configuration the first auxiliary connector (53) arranged on the pre-blood pump pre-dilution infusion line (21) is connected to the venous connector (41) and the second auxiliary connector (50) arranged on the pre-blood pump pre-dilution infusion line (21) is connected to the arterial connector (40).

According to a 59th aspect, depending on aspects 12 and 18, in said recirculation configuration the first auxiliary connector (57) arranged on the post-dilution fluid line (25) is connected to the arterial connector (40) and the second auxiliary connector (56) arranged on the post-dilution fluid line (25) is connected to the venous connector (41).

According to a 60th aspect, depending on aspects 12 and 18, in said recirculation configuration the first auxiliary connector (57) arranged on the post-dilution fluid line (25) is connected to the venous connector (41) and the second auxiliary connector (56) arranged on the post-dilution fluid line (25) is connected to the arterial connector (40).

According to a 61st aspect, depending on aspects 12, 15 and 20 or 21, in said recirculation configuration, the first auxiliary connector (59) arranged on the blood circuit (6, 7), in particular on the blood return line (7) is connected to said arterial connector (40) and the second auxiliary connector (58) arranged on the blood circuit (6, 7), in particular on the blood withdrawal line (6), is connected to the venous connector (41).

According to a 62nd aspect, depending on aspects 12 and 59 or on aspects 12 and 60, the at least one first pressure drop valve or flow resistor (60) is arranged on said post-dilution fluid line (25).

According to a 63rd aspect, depending on aspects 12 and 55 or on aspects 12 and 56, the at least one first pressure drop valve or flow resistor (60) is arranged on said post-blood pump pre-dilution infusion line (15).

According to a 63^{rd}-bis aspect, depending on aspects 12 and 57 or on aspects 12 and 58, the at least one first pressure drop valve or flow resistor (60) is arranged on said pre-blood pump pre-dilution infusion line (21).

According to a 64th aspect, depending on one or more of the previous aspects, the disposable circuit comprises at least one sensor for determining a fluid (e.g., blood) flow rate in at least the blood circuit (6, 7).

According to a further aspect depending on one or more of the previous aspects, the first and the second auxiliary connectors are separate connectors. Alternatively, the first and the second auxiliary connectors may be made in a single piece (e.g., a connector having 4 ways, two irremovably connected to the respective tube portions and the remaining two for connection to first and the second auxiliary connector respectively).

In a 65th aspect, according to the present invention is herewith disclosed a use of a disposable circuit according to one or more of aspects 1-64 in an apparatus for extracorporeal treatment of blood for chronic patients.

In a 66th aspect, according to the present invention is herewith disclosed the use of a disposable circuit according to one or more of the preceding claims in an apparatus for extracorporeal treatment of blood for acute patients.

In a 67th aspect, according to the present invention is herewith disclosed an apparatus for extracorporeal treatment of blood, comprising a disposable circuit according to one or more of aspects 1-64, said apparatus further comprising a blood pump (11) configured at least to control the flow of blood in said blood circuit (6, 7).

In a 67^{th}-bis aspect, an apparatus is provided comprising a disposable circuit according to one or more of aspects 1 to 64, the apparatus further comprising a control unit (10) and a blood pump (11) configured to control the flow of blood in said blood circuit (6, 7),

wherein the control unit (10) is configured to:
stop the blood pump (11) before patient disconnection;
receive a command of activation of a fluid recirculation in the blood circuit (6, 7);
when the disposable circuit is in a recirculation configuration, activate at least the blood pump (11), to cause recirculation of fluid inside the blood circuit (6, 7).

According to a 68th aspect, depending on aspects 67, the apparatus further comprises at least one flow-regulating actuator, in particular a fluid pump (18, 22, 27) for at least one of said infusion lines (15, 21, 25), optionally wherein one or more of the following lines is present:
a post blood pump pre-dilution infusion line (15), connected to the blood withdrawal line (6), the apparatus comprising at least one infusion pump (18) for regulating the flow of fluid through said post blood pump pre-dilution infusion line (15);
a post-dilution fluid line (25), connected to the blood return line, the apparatus comprising at least one infusion pump (27) for regulating the flow through said post-dilution fluid line (25);
a pre-blood pump pre-dilution infusion line (21), connected to the blood withdrawal line (6), the apparatus comprising at least one pre-blood infusion pump (22) for regulating the flow through said pre-blood pump pre-dilution infusion line (21).

According to a 68^{th}-bis aspect depending on aspects 67, in the recirculation configuration, the first auxiliary connector (51, 53, 55, 57, 59) is directly connected either with the arterial connector (40) or with the venous connector (41) and the second auxiliary connector (50, 52, 54, 56, 58) is directly connected with the other one of the arterial connector (40) and the venous connector (41), the control unit (10) being configured to maintain the disposable circuit in the recirculation configuration wherein during recirculation either a full flow rate of fluid entering into the first auxiliary connector (51, 53, 55, 57, 59) or into the second auxiliary connector (50, 52, 54, 56, 58) is split into separate partial flow rates or two partial flow rates of fluid entering into the first auxiliary connector (51, 53, 55, 57, 59) or into the second auxiliary connector (50, 52, 54, 56, 58) are merged and exit as a full flow rate.

According to a 69th aspect, depending on said aspects 67 or 68, at least one among the blood pump (11), the infusion pump (18) of the post blood pump pre-dilution infusion line (15), the infusion pump (27) of the post-dilution fluid line (25), the pre-blood infusion pump (22) of the post blood pump pre-dilution infusion line (21), the dialysis pump (21), the effluent liquid pump (17) is a peristaltic pump fixedly installed on the apparatus, and whose actuator is configured to force fluid motion through direct action, specifically a compression, on a pump tract of the respective line.

According to a 70th aspect, depending on one or more of aspects 67-69 and on aspect 47, the apparatus further comprises at least one scale selected in the group:
a first scale (34) operative for providing weight information relative to the amount of fluid supplied from said first infusion fluid container (16);
a second scale (36) operative for providing weight information relative to the amount of fluid supplied from said second infusion fluid container (23);
a third scale (37) operative for providing weight information relative to the amount of fluid supplied from said third infusion fluid container (26);
an effluent scale (33) operative for providing weight information relative to the amount of fluid collected in the effluent fluid container (14);
a dialysis scale (20) operative for providing weight information relative to the amount of fluid supplied by the dialysis fluid container (20).

According to a 71st aspect, depending on one or more of aspects 67-70, the apparatus further comprises a control unit (10) comprising at least one first operative configuration of blood wherein it is configured to:
receive or calculate initial values to one or more fluid flow rates selected in the group including a fluid flow rate (Q_{eff}) through the effluent line (13), a fluid flow rate (Qₚᵣₑ, Q_{pbp}, Qₚₒₛₜ) through the at least one infusion fluid line (15, 21, 25, 42; 42a), optionally through any of the post blood pump pre-dilution infusion line (15), pre-blood pump pre-dilution infusion line (21), post-dilution fluid line (25), a fluid flow rate (Q_{dial}) through the dialysis liquid fluid line (19), a fluid removal rate (Q_{pfr}) from the patient a blood flow rate (Q_{b}).

According to a 72nd aspect, depending on aspect 71, said control unit (10) is configured to actuate at least one among the blood pump (11), the infusion pump (18) of the post blood pump pre-dilution infusion line (15), the infusion pump (27) of the post-dilution fluid line (25), the pre-blood infusion pump (22) of the pre-blood pump pre-dilution infusion line (21), the dialysis pump (17a), the effluent liquid pump (17) to deliver the prescribed or calculated flow rate.

According to a 73rd aspect, depending on aspects 71 or 72 and 68, the control unit (10) comprises a second operative configuration, comprising:
receiving a command of activation of a temporary fluid recirculation in the blood circuit (6, 7);
set at least one value of recirculation flow rate through the blood circuit (6, 7);
activate at least the blood pump (11) to cause recirculation of fluid in the blood circuit (6, 7) when in the recirculation configuration;
wherein the second operative configuration takes place after the first operative configuration is interrupted and the flow rate of fluid in the blood circuit (6, 7) is reduced at least momentarily to zero by deactivating at least the blood pump (11), optionally the blood pump (11) and the infusion pump (18) of the pre-dilution fluid line (15), the infusion pump (27) of the post-dilution fluid line (25), the pre-blood infusion pump (22) of the pre-blood pump infusion line (21).

According to a 74th aspect, depending on one or more of said 71-73rd aspects, the control unit (10) is configured to obtain an electronic confirmation of disconnection of said arterial connector (40) and venous connector (41) before allowing the activation of the blood pump (11) for recirculation.

According to a 75th aspect, depending on one or more of aspects 63-74, the apparatus further comprises a safety clamp (9) fixedly arranged on the blood circuit (6, 7) comprising at least one open configuration allowing fluid flow through the blood circuit (6, 7) and a closed configuration preventing fluid flow through the blood circuit (6, 7), optionally wherein the safety clamp (9) is an electronically controlled safety clamp (9).

According to a 76th aspect, depending on one or more of aspects 72, 73, 74 when combined to aspect 75, the safety clamp (9) is operatively connected to the control unit (10), wherein in the first operative configuration and in the second operative configuration the safety clamp (9) receives a control signal causing its permanence in its open configuration or is in said open configuration and wherein when switching between the first operative configuration and the second operative configuration, the safety clamp (9) receives a control signal configuring it to the closed configuration.

According to a 77th aspect, depending on any of previous aspects, in the operative/treatment configuration the at least one first auxiliary connector (51, 53, 55, 57, 59), optionally the first auxiliary connector (51, 53, 55, 57, 59) and the second auxiliary connector (50, 52, 54, 56, 58), is/are not connected to the arterial connector (40) and the venous connector (41), and wherein in the recirculation configuration, alternative to the operative/treatment configuration the at least one first auxiliary connector (51, 53, 55, 57, 59) is connected with the arterial connector (40) or with the venous connector (41), optionally wherein the first auxiliary connector (51, 53, 55, 57, 59) is directly connected with one connector between the arterial connector (40) and the venous connector (41) and the second auxiliary connector (50, 52, 54, 56, 58) is directly connected with the other of the arterial connector (40) and the venous connector (41).

According to a 78th aspect, depending on aspect 68 and on any aspect depending therefrom, at least the blood pump (11), or the infusion pump (18) of the post-blood pump pre-dilution infusion line (15), or the infusion pump (27) of the post-dilution fluid line (25), or the pre-blood infusion pump (22) of the pre-blood pump pre-dilution infusion line (21), or the dialysis pump (17a), or the effluent liquid pump (17) is/are occlusive pumps, optionally peristaltic pumps.

According to a 79th aspect, is herewith disclosed a method of blood flow recirculation in a disposable circuit, said disposable circuit being provided with:
a filtration unit (2);
a blood circuit (6, 7) comprising a blood withdrawal line (6) having a first end connected to an inlet of the filtration unit (2), and a blood return line (7) having a first end connected to an outlet of the filtration unit (2), said blood withdrawal line (6) and said blood return line (7) being designed to be connected to a patient cardiovascular system,
wherein the blood withdrawal line (6) has a second end provided with an arterial connector (40) and the blood return line (7) has a second end provided with a venous connector (41), said arterial connector (40) and said venous connector (41) being designed to be detachably connected to a vascular access of a patient; and wherein said blood circuit is configured to be interfaced with a blood pump (11) for controlling the flow in the blood circuit (6, 7);

said method comprising the following steps:
stopping a blood pump (11) active on the blood circuit;
disconnecting the arterial connector (40 from an access to the patient cardiovascular system);
disconnecting the venous connector (41) from an access to the patient cardiovascular system;
configuring the disposable circuit in a recirculation configuration by connecting at least one first auxiliary connector (51, 53, 55, 57, 59) arranged on the blood circuit (6, 7) or arranged on at least one fluid line (15, 21, 25, 42; 42a) with said arterial connector (40) or said venous connector (41), wherein after said connection a closed circuit allowing fluid recirculation is defined, optionally wherein the step of configuring the disposable circuit in the recirculation configuration further comprises connecting a second auxiliary connector (50, 52, 54, 56, 58) arranged on the blood circuit or on said at least one fluid line (15, 21, 25, 42; 42a) with the other of said arterial connector (40) or said venous connector (41).

According to an 80th aspect is herewith disclosed a method of temporary disconnection of a patient from an apparatus for the extracorporeal treatment of blood, the apparatus comprises a disposable circuit having:
a filtration unit (2) optionally having a primary chamber (3) and a secondary chamber (4) separated by a semi-permeable membrane (5);
a blood circuit (6, 7) comprising a blood withdrawal line (6) having a first end connected to an inlet of the filtration unit (2), e.g., of the primary chamber (3), and a blood return line (7) having a first end connected to an outlet of the filtration unit (2), e.g., of the primary chamber (3), said blood withdrawal line (6) and said blood return line (7) being designed to be connected to a patient cardiovascular system,
wherein the blood withdrawal line (6) has a second end provided with an arterial connector (40) and the blood return line (7) has a second end provided with a venous connector (41), said arterial connector (40) and said venous connector (41) being designed to be detachably connected to a vascular access device of a patient; and wherein said blood circuit is configured to be interfaced with a blood pump (11) for controlling the flow in the blood circuit (6, 7);
at least one fluid line (15, 21, 25, 42; 42a) connected to the blood circuit (6, 7), the disposable circuit further comprising at least one first auxiliary connector (51, 53, 55, 57, 59) arranged either on the blood circuit (6, 7) or on said at least one fluid line (15, 21, 25, 42; 42a) and optionally a second auxiliary connector (50, 52, 54, 56, 58) arranged on the blood circuit or on said at least one fluid line (15, 21, 25, 42; 42a), the method comprising:
stopping a blood pump active on the blood circuit;
disconnecting the arterial connector (40) from the access device of the patient;
disconnecting the venous connector (41) from the access device of the patient;
connecting one of the arterial connector (40) and the venous connector (41) to the first auxiliary connector (51, 53, 55, 57, 59) to define a recirculation configuration;
optionally connecting the other one of the arterial connector (40) and the venous connector (41) to the second auxiliary connector (50, 52, 54, 56, 58) to define a closed circuit in the disposable circuit to define the recirculation configuration;
running the blood pump to start recirculating fluid inside the closed circuit when the patient remains disconnected from the apparatus.

Notably, the disposable circuit of aspect 80th is according to any one of the aspects dealing with the disposable circuit, e.g., 1-64.

According to an 80^{th}-bis aspect is herewith disclosed a method of configuration of a disposable circuit on an apparatus for extracorporeal treatment of blood, the method comprising:
arranging the disposable circuit according to any of the preceding aspects from 1 to 78 on an apparatus for blood treatment provided at least with a blood pump (11) configured to force fluid flow circulation at least into the primary chamber (3) of the filtration unit, such step of arranging the disposable circuit leading to an operative configuration wherein at least part of the blood circuit (6, 7) interacts with said blood pump (11), so that the first auxiliary connector (51, 53, 55, 57, 59), and the second auxiliary connector (50, 52, 54, 56, 58) are disconnected from the arterial (40) and venous connector (41)
configuring the disposable circuit in a recirculation configuration, by connecting the first auxiliary connector (51, 53, 55, 57, 59) arranged on the blood circuit (6, 7) or on the fluid line (15, 21, 25, 42; 42a) with said arterial connector (40) or said venous connector (41), wherein after said connecting a closed circuit allowing fluid recirculation is defined, optionally wherein the step of configuring the disposable circuit in the recirculation configuration further comprises connecting the second auxiliary connector (50, 52, 54, 56, 58) arranged on the blood circuit or on said fluid line (15, 21, 25, 42; 42a) with the other of said arterial connector (40) or said venous connector (41).

According to an 81st aspect, depending on one or more of the aspects 79 or 80, the method further comprises:
stopping the blood pump to stop recirculating fluid inside the closed circuit;
disconnecting the arterial connector (40) or the venous connector (41) from the first auxiliary connector (51, 53, 55, 57, 59);
optionally disconnecting the other one of the arterial connector (40) and the venous connector (41) from the second auxiliary connector (50, 52, 54, 56, 58);
reconnecting both the arterial connector (40) and the venous connector (41) to the access device of the patient before resuming the treatment,
   wherein the method steps are performed without changing the disposable circuit and/or disconnecting the disposable circuit from the apparatus.

According to an 81^{st}-bis aspect, depending on one or more of the aspects 79 or 80 or 81, the step of arranging the disposable circuit on said apparatus comprises arranging at least part of said disposable circuit in accordance to pathway provided on a graphic user interface of the apparatus, optionally so that the steps for arranging said disposable circuit on said apparatus is guided.

According to an 82nd aspect, depending on one or more of aspects 79-81, connecting at least the first auxiliary connector (51, 53, 55, 57, 59) arranged on the blood circuit (6, 7) or arranged on at least one fluid line (15, 21, 25, 42; 42a) with said arterial connector (40) or said venous connector (41), wherein after said connection a closed circuit allowing fluid recirculation is defined, comprises moving the arterial connector (40) or the venous connector (41) towards the first auxiliary connector (51, 53, 55, 57, 59).

According to an 83rd aspect, depending on one or more of aspects 79-82, connecting the second auxiliary connector (50, 52, 54, 56, 58) arranged on the blood circuit or on said at least one fluid line (15, 21, 25, 42; 42a) with one of said arterial connector (40) or said venous connector (41) which is not already connected to said first auxiliary connector (51, 53, 55, 57, 59) defining said closed circuit, comprises moving the arterial connector (40) or the venous connector (41) towards the second auxiliary connector (50, 52, 54, 56, 58).

According to an 84th aspect, depending on one or more of aspects from 79 to 83, between said step of disconnection of the arterial connector (40) and of the venous connector (41) from an infusion line and the step of configuring the disposable circuit in said recirculation configuration, the blood circuit is temporarily in an open circuit configuration.

According to an 84^{th}-bis aspect, depending on one or more of aspects from 79 to 83, the method comprises restitute at least part of the extracorporeal blood to the patient before starting recirculation.

According to the previous aspect it is further provided disconnecting one connector between the arterial connector (40) and the venous connector (41) from the vascular access device of the patient (in particular the arterial connector) and connecting the disconnected connector to a bag containing physiological fluid, the method further comprises activating the blood pump to restitute extracorporeal blood to the patient, once at least part of the extracorporeal blood is restituted, disconnecting the connector from the bag and connecting it to either the first or the second auxiliary connector.

According to the previous aspect, the method comprises to subsequently to blood restitution, disconnecting the other one of the arterial connector (40) and the venous connector (41) from the vascular access device of the patient (in particular the venous connector) and connecting it to the other one of the first and the second auxiliary connector.

According to aspect 84th-bis, the method comprises activating an infusion pump on the infusion line to push fluid contained in a bag associated with the infusion line into the blood circuit while the patient is still connected to the extracorporeal blood circuit to restitute blood to the patient (without withdrawing blood from the patient), optionally the blood pump being activated and synchronized with the infusion pump.

According to the previous aspect, the method comprises keeping at least one of the arterial connector and the venous connector connected to the vascular access device of the patient (and preferably both connectors) during blood restitution. Alternatively to keeping both arterial and venous connectors connected to the vascular access device of the patient during blood restitution, one connector among the arterial and venous connectors is disconnected from the vascular access device and connected either to the first auxiliary connector or to the second auxiliary connector and thereafter blood restitution is started. More specifically, this variant uses infusion pump and associated solution bag (used for the therapy); the arterial line is set in the recirculation step ‘immediately’. This is also a benefit of this option/variant to minimize handling steps (and risk of contamination) at the same time with no need of an ancillary fluid bag.

According to the previous two aspects, the method comprises, after blood restitution, disconnecting the venous connector from the vascular access device of the patient and connecting the venous connector to either the first or the second auxiliary connector.

According to the previous three aspects, the method comprises, after blood restitution, disconnecting the arterial connector from the vascular access device of the patient and connecting the arterial connector to either the first or the second auxiliary connector.

According to any one of the previous aspects up to aspect 84^{th}-bis, after recirculation and before restart of the treatment, a step of discharging the physiological fluid used during recirculation is provided. For example, a drain bag may be connected to the venous connector and the arterial connector may be reconnected to the patient vascular access device, the blood pump withdraws blood from the patient and pushes physiological fluid into the drain bag.

According to an 85th aspect, depending on any of aspects 79 to 84, the method further comprises a step of clamping at least one respective tube portion of said blood withdrawal and blood return line (6, 7), the step of clamping taking place before the step of disconnection of the arterial connector (40) and the venous connector (41) from the patient vascular access;
said method comprising a step of unclamping the blood withdrawal and blood return line, said unclamping taking place after defining the closed circuit in said recirculation configuration.

According to an 86th aspect, depending on aspect 85, said clamping is performed in correspondence of the second end of the blood withdrawal line (6) and in correspondence of the second end of the blood return line (7).

According to an 87th aspect, depending on aspect 85 or aspect 86, the step of clamping comprises clamping at least one tube portion of the blood withdrawal line (6) or of the blood return line (7), optionally a tube portion of the blood withdrawal line (6) with a first manual clamp and a tube portion of the blood return line (7) with a second manual clamp.

According to an 88th aspect, depending on one or more of aspects 79-87, said disposable circuit is arranged on an apparatus for extracorporeal circulation, and wherein at least the steps of disconnection of the arterial connector (40) and of the venous connector (41) from the vascular access and the step of configuring the disposable circuit in a recirculation configuration are performed without removing the disposable circuit from said apparatus for extracorporeal circulation,
said method comprising a step of stopping at least one blood pump (11) of the apparatus for extracorporeal circulation before the performing the step of disconnecting the arterial connector (40) and the venous connector (41) from the vascular access,
said method comprising a step of reactivating at least the blood pump (11) of the apparatus for extracorporeal circulation after the step of configuring the disposable circuit in the recirculation configuration has been performed.

According to an 89th aspect, depending on aspect 88, said step of stopping at least the blood pump (11) and the step of reactivating at least the blood pump (11) are performed through the action of a user, in particular on a user interface connected to the control unit (10), the control unit (10) managing the operation of at least the blood pump (11) and respectively involve the transmission of a stop signal to said blood pump (11) and the transmission of a reactivation signal to said blood pump (11).

According to a 90th aspect, depending on aspect 88 or 89, in concomitance with said step of stopping at least one blood pump (11) of the apparatus for extracorporeal circulation, a dialysis pump (17a) acting on an dialysis (19) connected to the inlet of the secondary chamber (4) of the filtration unit (2) and an effluent fluid pump (17) acting on an effluent line (13) connected to the outlet of the secondary chamber (4) are stopped or the filtration unit bypassed, before the performing the step of disconnecting the arterial connector (40) and the venous connector (41) from the infusion line.

Of course all infusion pumps infusing into the blood circuit (if infusion lines are present) are stopped as well.

According to a 91st aspect, depending on one or more of aspects 79-90, the first auxiliary connector (51, 53, 55, 57, 59), optionally both the first auxiliary connector (51, 53, 55, 57, 59) and the second auxiliary connector (50, 52, 54, 56, 58), is/are provided with at least one disconnectable port (70; 70a) configured to be connected to the venous connector (41) and/or the arterial connector (40), and wherein the step of configuring the disposable circuit in the recirculation configuration comprises connecting the disconnectable port (70; 70a) of the first auxiliary connector (51, 53, 55, 57, 59) to the disconnectable port (72) of the venous connector (41) or of the arterial connector (40).

According an 92nd aspect, depending on aspect 91, the step of configuring the disposable circuit in the recirculation configuration comprises connecting the disconnectable port (70; 70a) of the first auxiliary connector (51, 53, 55, 57, 59) to the disconnectable port (72) of the venous connector (41) and connecting the disconnectable port of the second auxiliary connector (50, 52, 54, 56, 58) to the arterial connector (40) or, vice-versa, comprises connecting the disconnectable port (70; 70a) of the first auxiliary connector (51, 53, 55, 57, 59) to the disconnectable port (72) of the arterial connector (40) and connecting the disconnectable port of the second auxiliary connector (50, 52, 54, 56, 58) to the venous connector (41).

According to a 93rd aspect, depending on aspects 79-92,
the first auxiliary connector (51, 53, 55, 57, 59) and the second auxiliary connector (50, 52, 54, 56, 58) have the respective detachable port (70) of the same type, male or female type; or
the first auxiliary connector (51, 53, 55, 57, 59) has the at least one detachable port (70) of a male type and the second auxiliary connector (50, 52, 54, 56, 58) has the at least one detachable port (70) of a female type; or
the first auxiliary connector (51, 53, 55, 57, 59) has the at least one detachable port (70) of a female type and the second auxiliary connector (50, 52, 54, 56, 58) has the at least one detachable port (70) of a male type;
and wherein the step of connecting the disconnectable port (70; 70a) of the first auxiliary connector (51, 53, 55, 57, 59) to the disconnectable port (72) of the venous connector (41) and connecting the disconnectable port of the second auxiliary connector (50, 52, 54, 56, 58) to the arterial connector (40), or vice-versa, connecting the disconnectable port (70; 70a) of the first auxiliary connector (51, 53, 55, 57, 59) to the disconnectable port (72) of the arterial connector (40) and connecting the disconnectable port of the second auxiliary connector (50, 52, 54, 56, 58) to the venous connector (41) determines a male-female type connection.

According to a 94th aspect, depending on one or more aspects 79-93, said one or more fluid lines (15, 21, 25; 42; 42a) comprise one or more of the following lines:
a post-blood pump pre-dilution infusion line (15), directly connected to the blood withdrawal line (6);
a pre-blood pump pre-dilution infusion line (21), directly connected to the blood withdrawal line (6);
a post-dilution fluid line (25), directly connected to the blood return line (7);
each among the post blood pump pre-dilution infusion line (15), the pre-blood pump pre-dilution infusion line (21) and the post-dilution fluid line (25) comprising a first end directly connected to the blood circuit (6, 7), and a second end, opposite to the first end, configured to be connected to, or connected to, a respective infusion fluid container (16, 23, 26), wherein in said recirculation configuration no fluid is withdrawn from the fluid container (16, 23, 26).

According to a 95th aspect, depending on one or more of aspects 79-94, the method further comprises a step of arranging at least one first pressure drop valve or flow resistor (60) configured for even fluid flow distribution during recirculation in the at least one fluid line (15, 21, 25, 42; 42a), optionally said method comprising a step of regulating the at least one first pressure drop valve or flow resistor (60) when said disposable circuit is in said recirculation configuration.

According to a 96th aspect, depending on aspects 79-95, the method comprises connecting at least the first auxiliary connector (51, 53, 55, 57, 59) arranged on the blood circuit (6, 7) or arranged on at least one fluid line (15, 21, 25, 42; 42a) with said arterial connector (40) or said venous connector (41), optionally connecting the second auxiliary connector (50, 52, 54, 56, 58) arranged on the blood circuit or on said at least one fluid line (15, 21, 25, 42; 42a) with the other of said arterial connector (40) and said venous connector (41), in particular comprises a direct connection of said first auxiliary connector (51, 53, 55, 57, 59) with said one connector between the arterial connector (40) or the venous connector (41), and a direct connection of the second auxiliary connector (50, 52, 54, 56, 58) with the other of said arterial connector (40) and said venous connector (41).

According to a 97th aspect depending on aspect 94, in said recirculation configuration fluid flows from said arterial connector (40) through said primary chamber (3) to said venous connector (41) or from the second end of the blood withdrawal line (6) to the first end of the blood withdrawal line (6) and from the first end of the blood return line (7) to the second end of the blood return line (7).

According to a 98th aspect, depending on aspect 94, the first and the second auxiliary connectors (50, 51) are arranged on the post-blood pump pre-dilution infusion line (15) and wherein in said recirculation configuration the first auxiliary connector (51) arranged on the post-blood pump pre-dilution infusion line (15) is connected to the venous connector (41) and the second auxiliary connector (50) arranged on the post-blood pump pre-dilution infusion line (15) is connected to the arterial connector (40).

According to a 99th aspect, depending on aspect 94, the first and the second auxiliary connectors are arranged on the post-blood pump pre-dilution infusion line (15) and wherein in said recirculation configuration the first auxiliary connector (51) arranged on the post-blood pump pre-dilution infusion line (15) is connected to the arterial connector (40) and the second auxiliary connector (50) arranged on the post-blood pump pre-dilution infusion line (15) is connected to the venous connector (41).

According to a 100th aspect, depending on aspect 94, the first and the second auxiliary connectors (52, 53) are arranged on the pre-blood pump pre-dilution infusion line (21) and wherein in said recirculation configuration the first auxiliary connector (53) arranged on the pre-blood pump pre-dilution infusion line (21) is connected to the arterial connector (40) and the second auxiliary connector (52) arranged on the pre-blood pump pre-dilution infusion line (21) is connected to the venous connector (41).

According to a 101st aspect, depending on aspect 94, wherein the first and the second auxiliary connectors (52, 53) are arranged on the pre-blood pump pre-dilution infusion line (21) and wherein in said recirculation configuration the first auxiliary connector (53) arranged on the pre-blood pump pre-dilution infusion line (21) is connected to the venous connector (41) and the second auxiliary connector (52) arranged on the pre-blood pump pre-dilution infusion line (21) is connected to the arterial connector (40).

According to a 102nd aspect, depending on aspect 94, wherein the first and the second auxiliary connectors are arranged on the post-dilution fluid line (25) and wherein in said recirculation configuration the first auxiliary connector (57) arranged on the post-dilution fluid line (25) is connected to the arterial connector (40) and the second auxiliary connector (56) arranged on the post-dilution fluid line (25) is connected to the venous connector (41).

According to a 103rd aspect, depending on aspect 94, the first and the second auxiliary connectors are arranged on the post-dilution fluid line (25) and wherein in said recirculation configuration the first auxiliary connector (57) arranged on the post-dilution fluid line (25) is connected to the venous connector (41) and the second auxiliary connector (56) arranged on the post-dilution fluid line (25) is connected to the arterial connector (40).

According to a 104th aspect, depending on one or more of aspects 79-103, the first and the second auxiliary connectors are arranged on the blood circuit (6, 7), and wherein in said recirculation configuration, the first auxiliary connector (59) arranged on the blood circuit (6, 7), in particular on the blood return line (7) is connected to said arterial connector (40) and the second auxiliary connector (58) arranged on the blood circuit (6, 7), in particular on the blood withdrawal line (6), is connected to the venous connector (41).

According to a 105th aspect, it is herewith disclosed computer program product, stored in at least one non-transitory memory support, said computer program product comprising software code portions suitable to be executed by at least one data processing unit, said software code portions being configured to cause the execution of at least:
providing at least one first selection item on a graphic user interface for configuring an apparatus for blood treatment in a treatment configuration, wherein in said treatment configuration undesirable matter is removed from the blood through the circulation of the blood in a blood circuit (6, 7) of a disposable circuit according to any of previous aspects 1-78 mounted on the apparatus and a second selection item for configuring the apparatus for blood treatment in a recirculation configuration,
determining activation and deactivation steps of a blood pump (11) provided on said apparatus for extracorporeal blood recirculation and acting on the blood circuit (6, 7), wherein following the selection of the recirculation configuration, a deactivation signal is transmitted for deactivating the blood pump (11);
said software code portion being further configured to cause the execution of a reactivation of the blood pump (11) following receipt of a confirmation command entered by a user, said command corresponding a confirmation that the disposable circuit is in said recirculation configuration.

According to a 106th aspect, depending on aspect 105, the deactivation signal is an electric control signal transmitted from a control unit (10) of said apparatus to the blood pump (11).

According to a 107th aspect, depending on aspect 106 or aspect 105, following the selection of the recirculation configuration, the software code portions cause the display of a visual guide on a screen of said apparatus aimed to help configuring the disposable circuit in said recirculation configuration, and then cause the apparatus to switch to a ‘waiting confirmation’ status ended when the confirmation command is entered by the user.

According to a 108th aspect, depending on one or more of the preceding aspects, a control unit (10), is configured to provide through a user interface, at least one selection item for selecting at least one of a plurality of treatment modes, said treatment modes comprising hemodialysis (HD), hemofiltration with pre-dilution (HFₚᵣₑ), hemofiltration with post-dilution (HFₚₒₛₜ), hemofiltration with both pre-dilution and post-dilution (HFₚᵣₑ₋ₚₒₛₜ), hemodiafiltration with pre-dilution (HDFₚᵣₑ), hemodiafiltration with post-dilution (HDFₚₒₛₜ), hemodiafiltration with both pre-dilution and post-dilution (HDFₚᵣₑ₋ₚₒₛₜ), ultrafiltration (UF), and to control the operation of at least said apparatus in accordance to the selected treatment mode.

### DESCRIPTION OF THE DRAWINGS

Aspects of the invention are shown in the attached drawings, which are provided by way of non-limiting example, wherein:

FIG. 1 shows a general schematic diagram of a disposable circuit according to an embodiment of the invention,

FIG. 2 and FIG. 3 show exemplary type of connection of auxiliary connectors of the disposable circuit according to an embodiment of the invention, when such auxiliary connectors are arranged on a pre-blood pump pre-dilution infusion line;

FIG. 4 and FIG. 5 show exemplary type of connection of auxiliary connectors of the disposable circuit according to an embodiment of the invention, when such auxiliary connectors are arranged on a post-blood pump pre-dilution infusion line;

FIG. 6 shows a specific and partial schematic diagram of a configuration of the disposable circuit;

FIG. 7 and FIG. 8 show exemplary type of connection of auxiliary connectors of the disposable circuit according to an embodiment of the invention, when such auxiliary connectors are arranged on a post-dilution fluid line;

FIG. 9 shows a specific and partial schematic diagram of a configuration of the disposable circuit;

FIG. 10 and FIG. 11 show alternative types of connection of auxiliary connectors of the disposable circuit according to an embodiment of the invention, when such auxiliary connectors are arranged on a blood circuit;

FIGS. 12, 13, and 14 show exemplary type of connection of auxiliary connectors of the disposable circuit according to an embodiment of the invention, when such auxiliary connectors are arranged on one, or two, auxiliary lines;

FIGS. 15-19 show alternative configurations for auxiliary connectors of the disposable circuit;

FIG. 20 shows a flow chart of some operative steps required for switching from a treatment configuration to a recirculation configuration for the apparatus and disposable circuit object of the present disclosure.

### DETAILED DESCRIPTION

Non-limiting embodiments of an apparatus 1 for extracorporeal treatment of blood—which may implement innovative aspects of the invention—are shown in figures. In below description and in figures, same components are identified by same reference numerals.

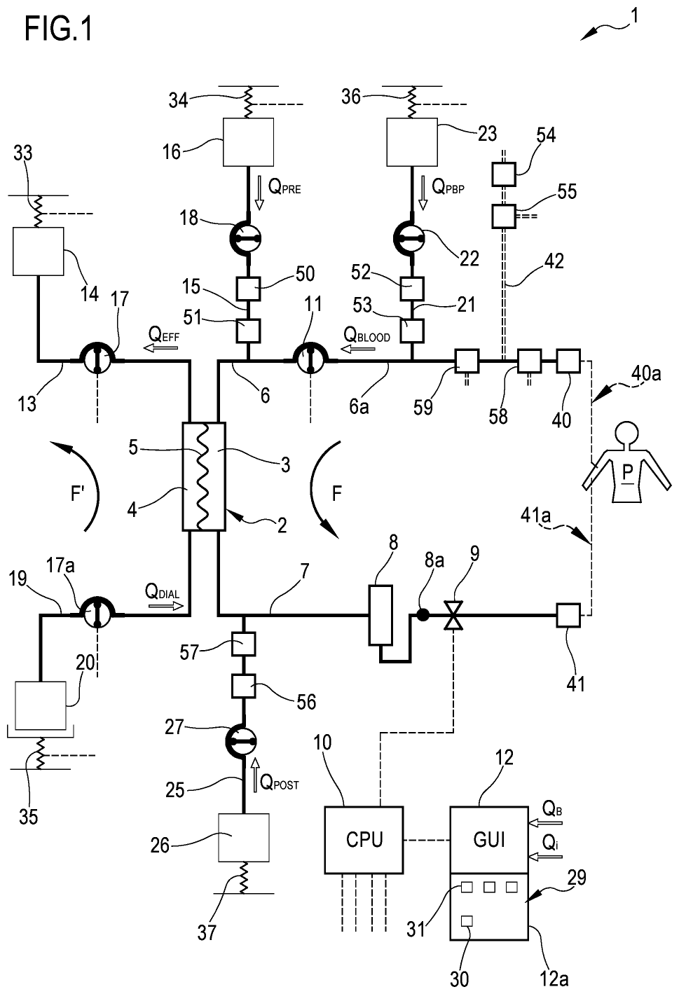

FIG. 1 shows an apparatus 1 designed for delivering extracorporeal blood treatments to patients, such as treatment for removing unwanted matter from the blood thereof.

According to further embodiments, an apparatus for extracorporeal blood treatment by hemoperfusion and/or or ExtraCorporeal CO2 Removal (ECCO2R) are also included in the scope of the invention.

In a non-limiting embodiment, the apparatus 1 is conceived for running of at least one of the following treatments: hemodialysis (HD), hemofiltration with pre-dilution (HFₚᵣₑ), hemofiltration with post-dilution (HFₚₒₛₜ), hemofiltration with both pre-dilution and post-dilution (HFₚᵣₑ₋ₚₒₛₜ), hemodiafiltration with pre-dilution (HDFₚᵣₑ), hemodiafiltration with post-dilution (HDFₚₒₛₜ), hemodiafiltration with both pre-dilution and post-dilution (HDFₚᵣₑ₋ₚₒₛₜ), ultrafiltration (UF). The apparatus may alternatively provide plasmafiltration.

The apparatus 1 comprises a disposable circuit for blood treatment, which comprises several elements hereinafter identified. For the purposes of the present invention, as per “disposable” shall be meant an element intended, specifically conceived, for being thrown away or anyway disposed after use, in particular after a single use. In particular it shall be noted that for “single use” shall be intended a single patient treatment. It is understood that the temporary disconnection of the patient from the apparatus 1, in the course of a non-completed treatment, does not interrupt the single treatment; reconnection of the patient to the disposable circuit after the temporary disconnection is yet still within the single use as herewith defined. The elements which according to any of the non-limiting embodiments hereinafter described are part of the disposable circuit, are marked in thick lines in the annexed figures; those being part of the apparatus 1 are marked in thin lines; this helps the reader to clearly identify which elements belong to the disposable circuit and which are in contrast part of the apparatus. The disposable circuit hereinafter described may be detachably connected to the apparatus and may be sold or anyway provided separately from the apparatus 1.

The disposable circuit comprises a filtration unit 2, such as an hemofilter, an ultrafilter, an hemodiafilter, a dialyzer, a plasmafilter, a gas exchanger, an adsorbent cartridge and the like; specifically, in a first example according to FIG. 1 particularly dealing with a dialysis apparatus, the filtration unit may have a primary chamber 3 and a secondary chamber 4 separated by a semi-permeable membrane 5; depending upon the treatment the membrane of the filtration unit may be selected to have different properties and performances.

The disposable circuit comprises a blood withdrawal line 6 connected to an inlet of the primary chamber 3, and a blood return line 7 connected to an outlet of the primary chamber 3. In use, the blood withdrawal line 6 and the blood return line 7 are connected to a vascular access device in communication with the patient vascular system. For example the blood withdrawal and return lines may be connected to a fixed catheter (e.g., a central venous catheter for acute patients) or to respective needles, in particular albeit in a non-limiting extent metal needles (to be inserted in e.g., an arteriovenous fistula for chronic patients), or other access device (not shown in annexed figures) which, in a first configuration of the disposable circuit herein described as “operative configuration” or “treatment configuration”, is placed in fluid communication with the patient vascular system, such that blood may be withdrawn through the blood withdrawal line, flowed through the primary chamber and then returned to the patient's vascular system through the blood return line. The blood return line 7 and the blood withdrawal line 6 are made of flexible plastic material. In a non-limiting solution, the blood withdrawal line 6 and the blood return line 7 are made of transparent plastic material, allowing to identify presence of blood.

In more detail, the blood withdrawal line 6 comprises a first end connected to the inlet of the primary chamber 3 and a second end opposite to the first end connected to an arterial connector 40, which is the last part of the disposable circuit an placed downstream the arterial access 40a providing access to the vascular system of the patient “P”; the blood return line 7 comprises a first end connected to the outlet of the primary chamber 3 and a second end opposite to the first end connected to a venous connector 41, which is the last part of the disposable circuit upstream the venous access 41a of the patient “P”.

In the course of the present disclosure reference will made to “downstream” and “upstream” for indicating the position of elements with respect to others. As marked in FIG. 1, with arrow F, the normal flow of fluid imposed by the blood pump 11 is from the arterial connector 40 to the venous connector 41; thus wording “downstream” and “upstream” will be referred considering such flow. Thus in the operative configuration the fluid moves from the arterial connector 40 through the portion 6a of the blood withdrawal line 6 up to the blood pump 11 that forces the flow at the inlet of the primary chamber 3 and then through the filter 2 up to the outlet of the primary chamber 3. The fluid then moves into the blood return line 7 up to its second end connected to the venous connector 41. For instance, the blood pump 11 lies upstream the primary chamber 3, and the outlet of the primary chamber 3 is downstream the inlet of the primary chamber 3 and/or the blood pump 11.

The arterial connector 40 and the venous connector 41 are non-disconnectable connectors, which are provided with a port fixed (e.g., welded or glued) to a respective tube portion (the blood withdrawal line and the blood return line), and a free port which is detachably connectable to the respective arterial access 40a or venous access 41a (for example the arterial and venous needles or the catheter). The free port of the arterial and venous connector may be of a male type or of a female type. Preferably, the free ports of both the arterial and venous connectors 40, 41 are of a same type. FIGS. 18 and 19 show non-limiting and schematic embodiments of arterial or venous connectors whose free port 72 is of a female type (FIG. 18) or of a male type (FIG. 19). Luer connectors are preferably used. Being of the same connection type, the arterial connector 40 and the venous connector 41 cannot be directly connected together.

At least one air separator, such as a bubble trap 8, is present in the blood circuit 6, 7, and preferably on the blood return line (a further bubble trap may be provided in the withdrawal line, too); moreover, a safety clamp 9 of the apparatus is active on the blood return line, and it is usually placed downstream the bubble trap 8 and close to the venous connector 41. The safety clamp 9 may be controlled by a control unit 10 of the apparatus 1. It shall be noted that albeit an air separator and a safety clamp are optional elements, their presence is preferable.

Additionally, the apparatus further comprises a bubble sensor 8a, active in correspondence of a portion of the return line 7 between bubble trap 8 and safety clamp 9; the bubble sensor is connected to the control unit 10 and sends to the control unit signals in case one or more bubbles are detected; in such a case, the control unit may stop the treatment and cause closure of the clamp 9. A corresponding safety clamp of the apparatus (not represented) may be provided in correspondence of the blood withdrawal line 6. The safety clamp is active on the blood withdrawal line 6 immediately downstream the arterial connector 40.

As shown in FIG. 1, the blood flow through the blood lines is controlled by a blood pump 11, for instance a peristaltic blood pump, acting either on the blood withdrawal line or on the blood return line. The blood pump 11 is a peristaltic blood pump, part of the apparatus 1. An operator may enter a set value for the blood flow rate Q_{B} through a user interface 12 (or the apparatus may calculate the blood flow rate based on other set parameters) and the control unit 10, during treatment, is configured to control the blood pump based on the set/calculated blood flow rate. In the course of the present description it is indicated that the control unit is “configured” or “programmed” to execute certain steps: this may be achieved in practice by any means which allow configuring or programming the control unit.

The apparatus 1 is provided with a (graphic) user interface which is configured to allow setting parameters for the treatment and to impart commands and controls to the apparatus 1 in the various operative configurations thereof. The user interface may comprise a screen or monitor, optionally touch sensitive, and one or more switches and/or pushbuttons specifically configured to cause a particular operation of the apparatus 1.

The disposable circuit further comprise an effluent fluid line 13 connected, at one end, to an outlet of the secondary chamber 4 and, at the other end, to an effluent fluid container 14 collecting spent fluid extracted from the secondary chamber. Alternatively the effluent line 13 may direct spent fluid directly to a drain. The effluent fluid line is configured to be interfaced with an effluent fluid pump 17 of the apparatus: the effluent fluid pump 17 operates on the effluent fluid line under the control of the control unit 10 to regulate the flow rate Q_{eff} across the effluent fluid line.

A dialysis fluid line 19 is connected at one end with a dialysis fluid container 20 (or to a dialysis fluid preparation device) and at its other end with the inlet of the secondary chamber 4 of the filtration unit. The dialysis fluid line 19 of the fluid circuit is configured to cooperate with a dialysis liquid pump 17a of the apparatus 1. The dialysis liquid pump 17a works on the dialysis liquid fluid line under the control of said control unit 10, to supply fresh dialysis fluid from the dialysis liquid container to the secondary chamber at a flow rate Q_{dial}.

According to certain embodiments for acute treatment, disposable circuits may comprise the above mentioned effluent fluid line 13 and dialysis fluid line 19 as disposable components integrally fixed to the filtration unit. At the end of the treatment, the entire disposable set is disposed. The dialysis fluid line 19 received fresh fluid from container 20, the spent dialysis fluid is routed to the effluent bag 14.

Differently, chronic renal treatment disposables include neither the dialysis fluid line nor the effluent fluid line as disposable parts. The dialysis fluid line and the effluent fluid line are non-disposable fluid paths arranged into the apparatus 1. A detachable connection is provided to removably place in fluid communication the dialysis fluid line and the effluent fluid line respectively to the inlet and the outlet of the secondary chamber 4 of the filtration unit; the latter being part of the disposable circuit. Dialysis fluid is either on-line prepared mixing water with concentrates, or received from a central dialysis fluid preparation plant.

In the context of the present disclosure, wording “upstream” and “downstream” will be referred in accordance to the fluid flow imposed by the effluent fluid pump 17 and by the dialysis pump 17a, which in FIG. 1 is marked by the arrow F′. Effluent fluid pump 17 and dialysis pump 17a cooperate to force fluid flow from the dialysis fluid container 20 through the dialysis fluid line 19, to the dialysis pump 17a, then to the inlet of the secondary chamber 4 through the filter 2 up to the outlet of the secondary chamber 4, when fluid enters the effluent fluid line 13 to flow at the effluent fluid pump 17 and then to the effluent fluid container 14.

The dialysis fluid pump 17a and the effluent fluid pump 17 are part of means for regulating the flow of fluid through the respective lines and, as mentioned, are operatively connected to the control unit 10 which controls the pumps. The control unit 10 is also connected to the user interface 12, for instance a graphic user interface, which receives operator's inputs and displays the apparatus outputs. For instance, the graphic user interface 12 may include a touch screen, a display screen and hard or soft keys for entering user's inputs or a combination thereof.

The disposable circuit shown in FIG. 1 may include an infusion line 21 connected, at one end, with a portion 6a of the blood withdrawal line 6 positioned upstream the blood pump 11 and, at its other end, with an infusion fluid container 23, which for instance may contain a regional anticoagulant such as a citrate solution, or a replacement solution or other fluids. This infusion line is hereinafter referred to as pre-blood pump pre-dilution infusion line 21. The means for regulating the fluid flow comprises a pump 22, for instance a peristaltic pump controlled by control unit 10, acting on a segment of the pre-blood pump pre-dilution infusion line 21 to regulate a pre-blood pump infusion rate Q_{pbp}. The pump 22 is part of the apparatus 1.

For the purposes of the present disclosure the three way connector above described is a “non-disconnectable connector”, that means that in the normal use the connector is not configured to be disconnected from any portion of conduit connected at any of its three ports or, equivalently, that the portion of conduits connected at any of its three ports are fixedly jointed thereto, e.g. through bonding or sealing.

The embodiment of FIG. 1 also includes a post-blood pump pre-dilution fluid line 15 connected, at one end, directly with a portion 6a of the blood withdrawal line 6 positioned downstream the blood pump 11 and, at its other end, with a further infusion fluid container: this post-blood pump pre-dilution fluid line 15 supplies replacement fluid from an infusion fluid container 16 connected at one end of the post-blood pump pre-dilution fluid line 15. The apparatus further comprises an infusion pump 18, for instance a peristaltic pump, operating on the post-blood pump pre-dilution fluid line 15 to regulate the flow rate Qₚᵣₑ through the line 15.

When referring to the post blood pump pre-dilution infusion line 15, or to the pre-blood pump pre-dilution infusion line 21, wording “upstream” and “downstream” will be referred to the normal fluid flow during the operative configuration. Fluid flows from the container 16, 23, through the portion of conduit line on which the pump 18, 22 of any of the infusion lines acts, up to the non-disconnectable connector which connects the infusion line with the blood withdrawal line 6.

Alternatively to, or in combination with the pre-dilution fluid line 15, the apparatus of FIG. 1, may also present a post-dilution fluid line 25 connected at one end with a further container 26 of infusion liquid and connected at its other end with the blood return line 7. A further pump 27, for instance a peristaltic pump, may act under the control of control unit 10 on the post-dilution fluid line 25 and thus also be part of said means for regulating the flow through the fluid lines.

When referring to the post-dilution fluid line 25, wording “upstream” and “downstream” will be referred to the normal fluid flow during the operative configuration. Fluid flows from the container 26, through the portion of conduit line on which the pump 27 acts, up to the non-disconnectable connector which connects the infusion line with the blood return line 7.

Alternatively or in combination with the aforementioned fluid lines, the apparatus of FIG. 1 may comprise one or more auxiliary lines, which are connected, at one end, with the blood circuit, for example to the blood withdrawal line 6. FIG. 1 shows a single auxiliary line 42. A further pump, not shown in FIG. 1, for instance a peristaltic pump, may act under the control of control circuit 10 on the auxiliary line and thus also be part of said means for regulating the flow through the fluid lines.

When referring to the any of the auxiliary lines, wording “upstream” and “downstream” will be referred to the normal fluid flow which flows from the free end of the auxiliary line to the end which is connected to the blood circuit 6, 7.

The apparatus 1 may also comprise a first scale 33 operative for providing weight information relative to the amount of the spent fluid collected in the effluent fluid container 14; a second scale 34 operative for providing weight information relative to the amount of the fluid supplied from the infusion fluid container 16 via a post blood pump pre-dilution infusion line 15; a third scale 35 operative for providing weight information relative to the amount of the fluid supplied from dialysis fluid container 20. In case more infusion lines would be present, as a pre-blood pump pre-dilution infusion line 21, post-dilution fluid line 25, then a respective scale 36 and 37 could be present to provide weight information relative to the amount of fluid supplied from infusion containers 23, 26. The scales are all connected to the control unit 10 and provide said weight information for the control unit 10 to determine the actual quantity of fluid in each container and/or the actual flow rate of fluid supplied by or received in each container. The control unit 10 may then be configured to receive treatment selection information and check if the user entered the required dialysis parameters. The control unit may also be configured to receive weight information from the first scale and, depending upon the selected treatment, from the scales associated to the container supplying the lines involved in the delivery of the selected treatment) and to control, at least at the beginning of the treatment, the flow rate of at least one of the effluent fluid, the infusion fluid, the dialysis fluid by controlling said means for regulating based on said weight information, and the initial set values.

From a structural point of view one or more, optionally all the containers 14, 16, 20, 23, 26 may be disposable plastic containers, preferably soft plastic bags which are hang on a support carried by the respective scale. The means for regulating flows typically comprises pumps, although other regulating means as valves or combinations of valves and pumps could be used. The scales may comprise piezoelectric sensors, or strain gauges, or spring sensors, or any other type of transducer able to sense forces applied thereon. Although the examples in the figures show use of scales for determining the amount of fluid in the respective containers and for allowing calculation of the respective flow rates through the various lines, it should be noted that the above described aspects of the invention are compatible also with blood treatment machines using volumetric sensors for determining flow rates or combinations of mass and volumetric sensors.

Though the previous description mainly referred to dialysis apparatus, embodiments of the present disclosure further encompasses hemoperfusion. Hemoperfusion is a method of filtering the blood extracorporeally to remove one or more toxins. As with other extracorporeal methods, the blood travels from the patient into a blood withdrawal line 6, gets filtered in a filtration unit 2, and then travels back into the patient through a blood return line 7, typically by using venovenous access. In hemoperfusion, the blood perfuses a filter composed of artificial cells filled with activated carbon or another microporous material. In an hemoperfusion apparatus, no infusion line may be present. In such a case, the first and second auxiliary connectors are placed on the blood line, for examples in the positions as below disclosed according to the fourth embodiment. As a possible further hemoperfusion configuration, a pre-blood pump pre-infusion line may be included for injecting a solution into the blood upstream the blood pump. In this latter embodiment, the first and the second auxiliary connectors may be placed on the pre-blood pump pre-infusion line, or may remain on the blood circuit (or one connector may be placed on the pre-blood pump pre-infusion line and the other one may be placed on the blood line.

Further, the apparatus of embodiments may be (alternatively or in addition) an extracorporeal CO₂ Removal (ECCO2R) device; the blood line circuit may be connected to a CO₂ removal filtration unit 2 and the apparatus may or may not include any infusion lines. The first and second auxiliary connectors may be placed on the blood circuit in case no infusion line is available, or on either the blood circuit or the infusion line, if present.

The disposable circuit of the present disclosure is designed to allow for temporary disconnection and then patient reconnection to the same disposable circuit to continue a (temporary) interrupted treatment. To achieve the task, the disposable set is reconfigurable for allowing fluid (e.g., blood) recirculation mainly in the blood circuit. Alternatively to the treatment (operative) configuration above described, another configuration for the disposable circuit herewith described is here referred as “recirculation configuration”. In said configuration, the patient is disconnected from the disposable circuit and fluid therein present may be recirculated for allowing the patient to move away from the extracorporeal blood treatment apparatus. Thanks to this aspect, a patient may be temporarily disconnected from the apparatus 1 to perform or undergo other exams or treatments while the treatment performed with the apparatus 1 is suspended in a very easy way, without requesting expensive complete change of disposable circuit and with a reduced health risk in relation to the disconnection-recirculation-reconnection operations. Further, no additional components are required to achieve temporary disconnection, such as additional ‘Y’ connector, saline bag, etc. An easy switching or reconfiguration capability between the operative configuration and the recirculation configuration further eases the activity of the qualified operators that help the patient throughout the performance of the treatment. During the recirculation configuration, i.e. after the control unit 10 has been properly set for the recirculation configuration, the target means values of flow rate(s) and/or prescribed does is interrupted, average values that may have been calculated and/or measured by the control unit 10 or any of the sensors operatively connected thereto are preferably stored in a memory. Calculation and/or measurement thereof, starts back at the end of the temporary disconnection of the patient, when the control unit 10 is properly set for re-establishing the original operative configuration of blood treatment; optionally, albeit preferably, the control unit 10 may be configured to allow calculation and/or measurement of partial values before the instauration of the recirculation configuration and after the re-instauration of the operative configuration.

For this purpose, several embodiments of the disposable circuit have been conceived as being provided with a first and a second auxiliary connector, which in the recirculation configuration are connected one with the venous connector 40 and the other with the arterial connector 41 for the purposes of defining a properly closed circuit allowing the fluid recirculation under the action of the blood pump 11. Albeit in the detailed disclosure of the embodiments always two auxiliary connectors are disclosed, in line of principle at least one auxiliary connector could be sufficient to make the closed circuit. Indeed, though not preferred, it is possible to connect one blood line end (venous or arterial connector) to the first auxiliary connector and to leave the other blood line end as a dead end (the second blood end, namely the dead end, is simply clamped). The fluid inside the closed loop may then be recirculated. Preferably, saline or other replacement fluid should be recirculated (i.e., not blood).

Preferably, but in a non-limiting extent, the auxiliary connectors are three way connectors having a main inlet and a main outlet receiving pieces of the main line where they are placed and are provided with at least one disconnectable port which may be sealed through a cap or, alternatively, which may be of a self-sealing type. This latter type is such that the port automatically seals when another connector is disconnected from the disconnectable port of the auxiliary connector and automatically opens when the other connector is connected to the disconnectable port of the auxiliary connector. A self-sealing type auxiliary connector helps preventing unwanted fluid (in particular blood) leakage when the patient treatment is interrupted and the configuration of the disposable circuit is switched between the operative configuration and the recirculation configuration, reduces the risk of inclusion of air bubbles in the blood circuit and further eases the operations performed by the qualified operators that assist the patient.

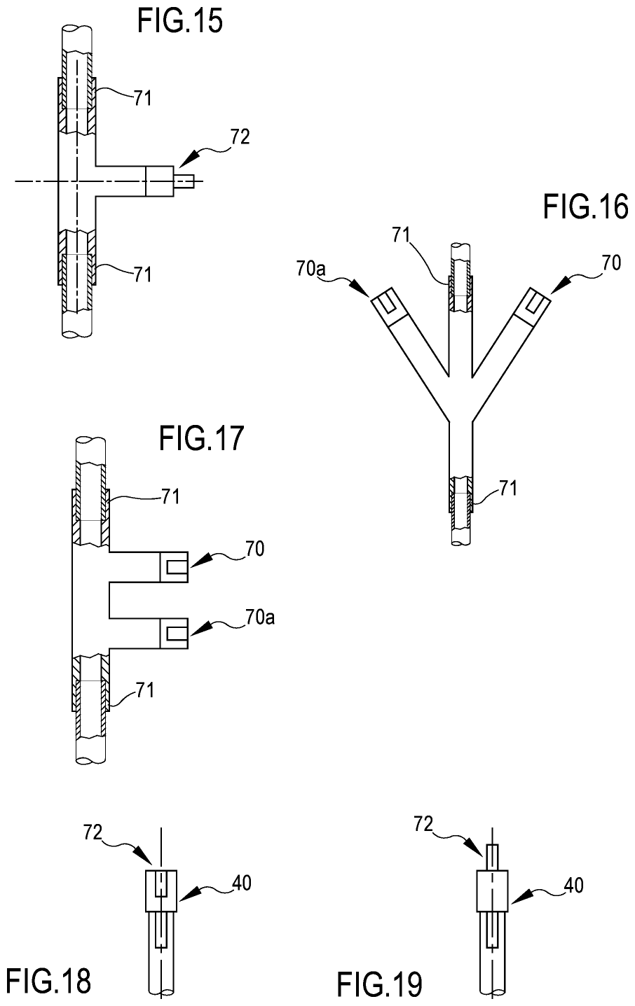

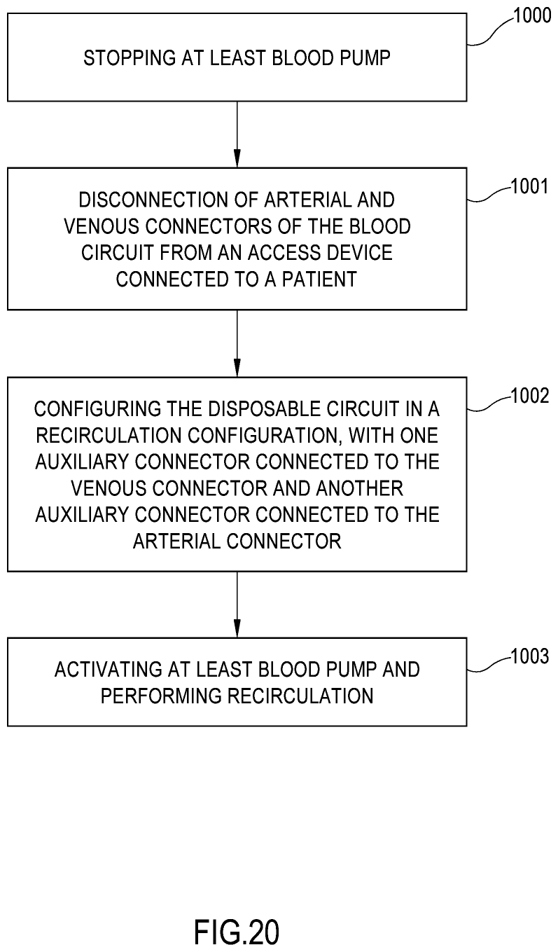

The auxiliary connector may have a wide variety of shapes, and may be provided with at least one first, or even with a second, disconnectable and free port which may be used to connect the auxiliary connector with another auxiliary connector and/or with the other one of the venous and the arterial connector. Non-limiting configurations of the connectors according to the present disclosure comprise three way auxiliary connectors like a “T” shaped connector (FIG. 15).

The illustrated connector (being it the first or the second auxiliary connector) comprises a first and a second non-disconnectable service port 71 fixed (e.g., sealed) to respective flexible tube portions. For example, the tube portions may be respective tube portions of the pre-blood pump pre-dilution line 21; alternatively, the tube portions may be respective tube portions of any other infusion line or tube portions of the blood withdrawal or return line. The disconnectable port 72 represented only schematically may be a male (or female) Luer connector configured to couple with the corresponding female (or male) Luer connector 40, 41 of the blood withdrawal line 6 or of the blood return line 7. Obviously a “Y” shaped connector, a half-snowflake shaped connector or another connector of different geometry may be used.

Looking at FIG. 16, the first and second auxiliary connectors may be made in a single piece, wherein the first and the second non-disconnectable service port 71 are still fixed (e.g., sealed) to respective flexible tube portions, but the merged connector includes two disconnectable ports 70, 70a for receiving the withdrawal line connector 40 and the return line connector 41, respectively. Also in this design, if the withdrawal line connector 40 and the return line connector 41 are Luer male connectors, then the two disconnectable ports 70 are Luer female connectors and vice versa.

The geometry of the ‘integral’ connector may also be different, such as the embodiment of FIG. 17, wherein the two disconnectable ports 70 emerge parallel to each other on the same lateral side of the device.

FIG. 1 presents, in a single schematic diagram, principal and non-limiting embodiments conceived by the inventors that are further herewith described in detail.

### First Embodiment

In a first embodiment the first auxiliary connector 53 and the second auxiliary connector 52 are arranged on the pre-blood pump pre-dilution infusion line 21, in a portion of conduit that once the disposable circuit is properly installed on the apparatus 1, lies downstream the pump 22 arranged on the pre-blood pump pre-dilution infusion line 21. The first auxiliary connector 53 is arranged downstream the second auxiliary connector 52.

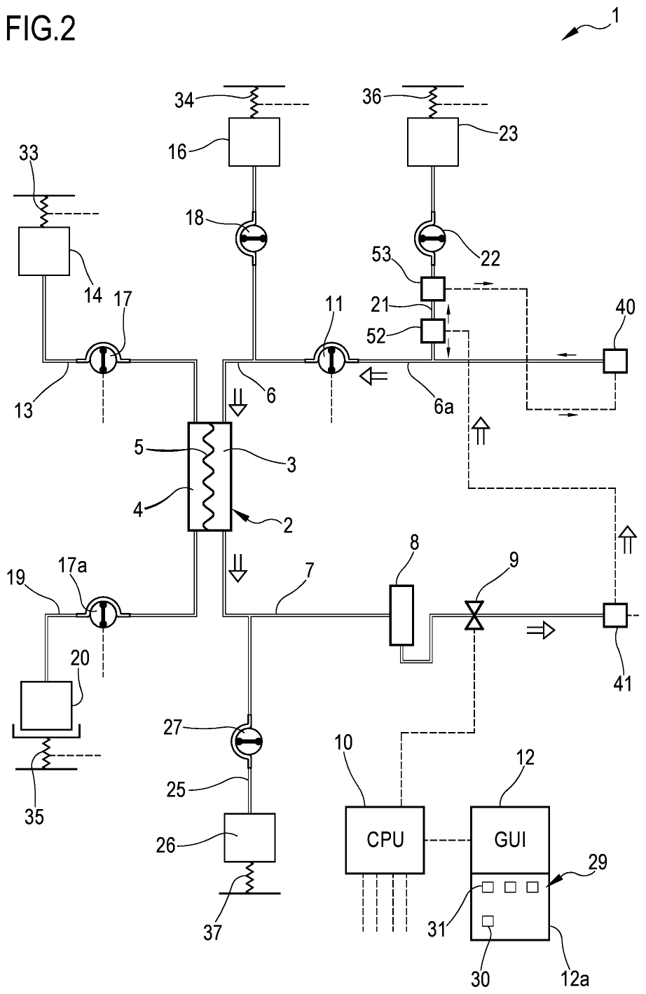

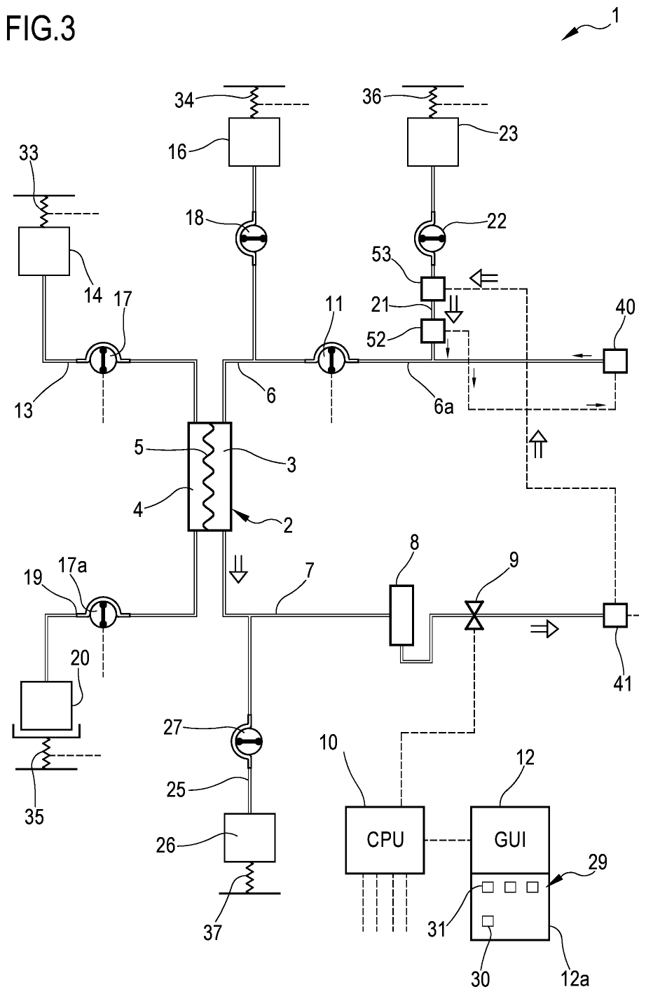

In the recirculation configuration, alternatively:
the first auxiliary connector 53 is connected to the arterial connector 40 and the second auxiliary connector 52 is connected to the venous connector 41 (FIG. 2); or
the first auxiliary connector 53 is connected to venous connector 41 and the second auxiliary connector 52 is connected to the arterial connector 40 (FIG. 3).

In the recirculation configuration of FIG. 2, full flow rate (double arrow) is present in the blood withdrawal line 6 downstream the pre-blood pump pre-dilution infusion line 21 and in the blood return line 7; a full flow rate arrives to the second auxiliary connector 52 and there the flow rate is split into partial flow rates (single arrows) that are present in the portions of lines connecting the second auxiliary connector 52 to the first auxiliary connector 53 and then in the portion of line that connects the arterial connector 40 to the pre-blood pump pre-dilution infusion line 21 injection point in the blood withdrawal line 6. Here the two partial flow rates merges again generating a full flow rate.

In FIGS. 2 and 3, a double arrow indicates a full flow rate and its direction along the lines; a single arrow indicates a split (partial) flow rate and its direction along the line portion. The same representation is used in respect to all the figures showing recirculation.

In the first embodiment the first and the second auxiliary connectors are both three-way connectors which are provided each with a disconnectable port 70 configured to be connected to the disconnectable port 72 of one connector selected between the venous connector 40 and the arterial connector 41 in accordance to one of the two possible alternatives above identified. The disconnectable port 72 of the first and second auxiliary connectors 53, 52 are of a type which opposite to the type of the disconnectable port of the venous and arterial connectors. In other words, when the disconnectable port 72 of the first and second auxiliary connectors 53, 52 are of a male type, the disconnectable port of the arterial and venous connector is of a female type, and vice-versa.

### Second Embodiment

In a second embodiment the first auxiliary connector 51 and the second auxiliary connector 50 are arranged on the post-blood pump pre-dilution infusion line 15, in a portion of conduit that once the disposable circuit is properly installed on the apparatus 1, lies downstream the pump 18 arranged on the post-blood pump pre-dilution infusion line 15. The first auxiliary connector 51 is arranged downstream the second auxiliary connector 50.

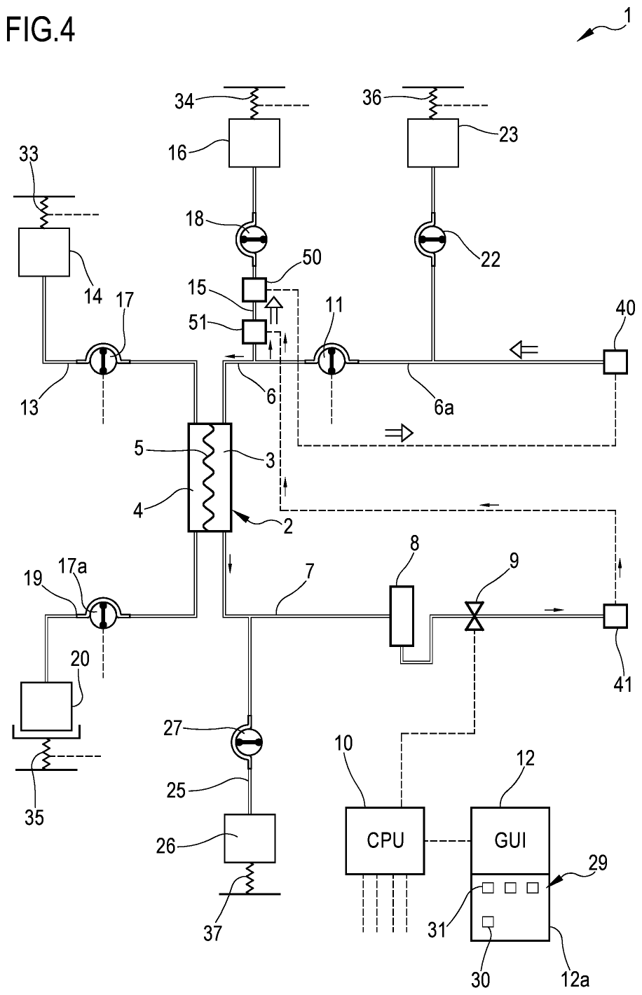

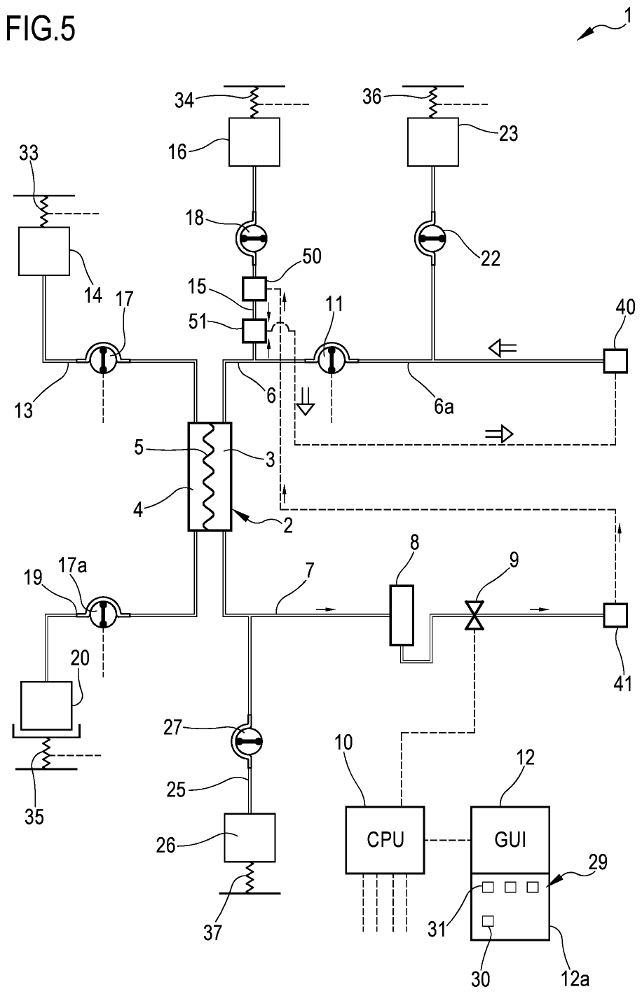

In the recirculation configuration, alternatively:
the first auxiliary connector 51 is connected to venous connector 41 and the second auxiliary connector 50 is connected to the arterial connector 40 (FIG. 4); or
the first auxiliary connector 51 is connected to the arterial connector 40 and the second auxiliary connector 50 is connected to the venous connector 41 (FIG. 5).

In the recirculation configuration of FIG. 4, full flow rate is present in the blood withdrawal line 6 upstream the post-blood pump pre-dilution infusion line 15 and, the full flow rate is split at the injection point of line 15 and partial flow rate is present in the blood return line 7, in the venous connector 41 up to the first auxiliary connector 51; here the partial flow coming up from the blood withdrawal line and the partial flow at the to the first auxiliary connector 50 merge and in the portion of line that connects the first auxiliary connector 51 to the second auxiliary connector 50 and up to the blood withdrawal line 6 there is full flow, as represented.

In the second embodiment the first and the second auxiliary connectors 50, 51 are both three-way connectors which are provided each with a disconnectable port 70 configured to be connected to another disconnectable port 72 of one connector selected between the venous connector 40, and the arterial connector 41 in accordance to one of the two possible alternatives above identified. The disconnectable port 72 of the first and second auxiliary connectors 51, 50 are of a type which opposite to the type of the disconnectable port of the venous and arterial connectors. In other words, when the disconnectable port 72 of the first and second auxiliary connectors 51, 50 are of a male type, the disconnectable port of the arterial and venous connector is of a female type, and vice-versa.

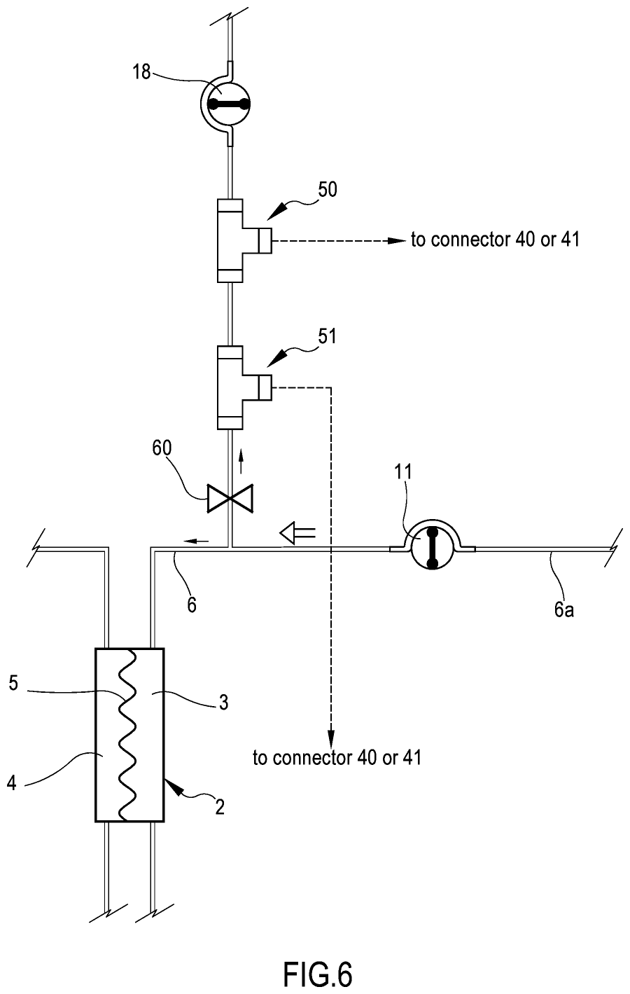

As schematically disclosed in FIG. 6, optionally, the post-blood pump pre-dilution infusion line 15 may comprise a pressure drop valve or flow resistor 60, which is in particular and in a non-limiting extent installed downstream the first auxiliary connector 51, just upstream the non-disconnectable three way connector that connects the post-blood pump pre-dilution infusion line 15 to the blood withdrawal line 6. The pressure drop valve or flow resistor 60 is configured to even flow resistances during recirculation and may be detachable or fixedly installed on the fluid line; optionally, the drop valve or flow resistor may be of a variable-resistance type. The aim of the drop valve or flow resistor 60 is to make the resistances of the respective portions of the pipe similar so that the flows generated in each of the circuit branches are as similar as possible.

### Third Embodiment

In a third embodiment the first auxiliary connector 57 and the second auxiliary connector 56 are arranged on the post-dilution fluid line 25, in a portion of conduit that once the disposable circuit is properly installed on the apparatus 1, lies downstream the pump 27 arranged on the post-dilution fluid line 25. The first auxiliary connector 57 is arranged downstream the second auxiliary connector 56.

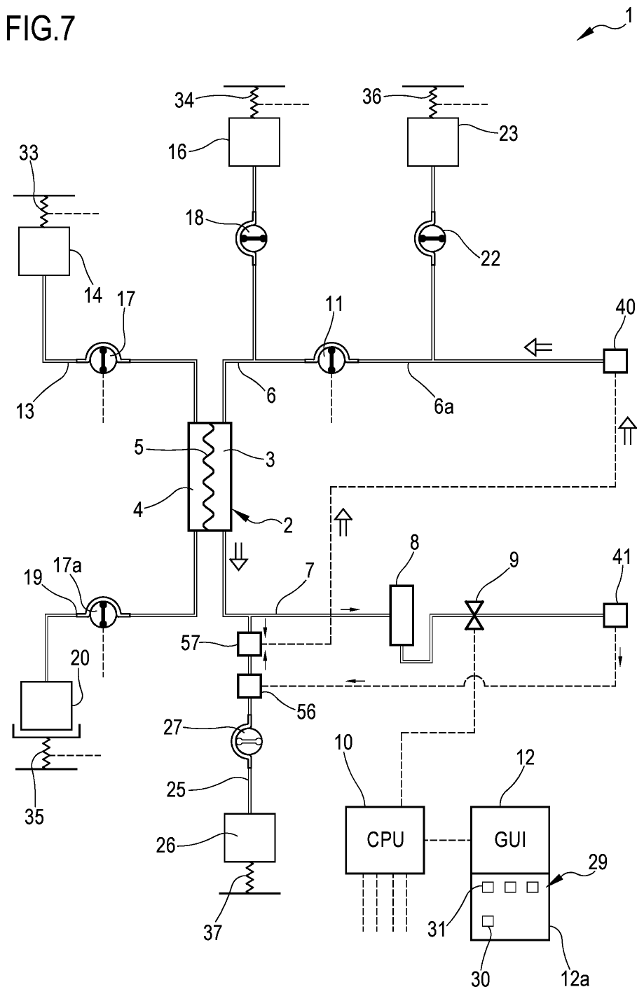

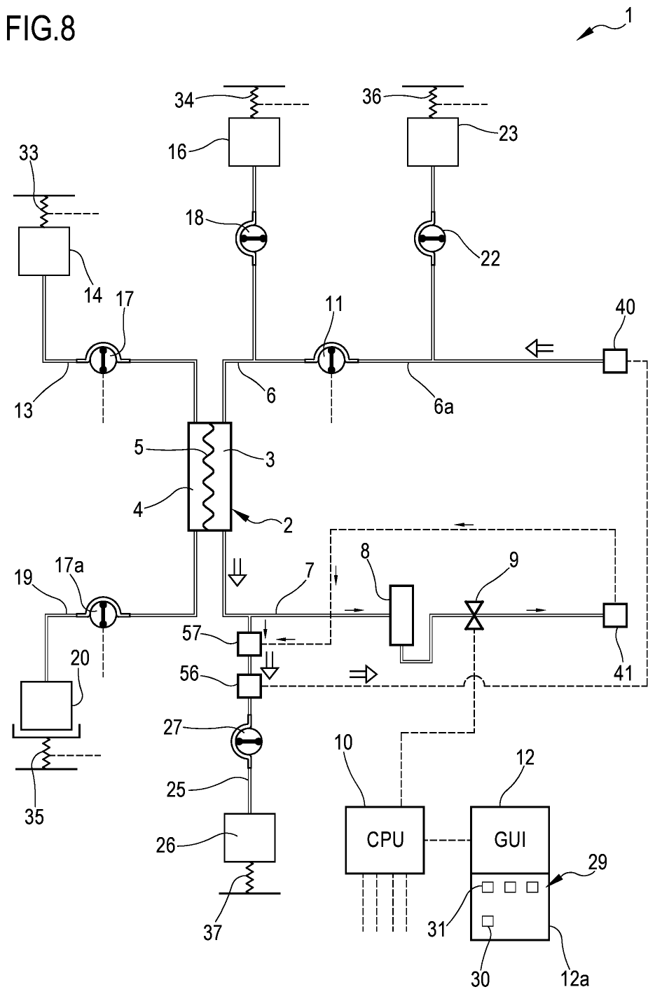

In the recirculation configuration, alternatively:
the first auxiliary connector 57 is connected to the arterial connector 40 and the second auxiliary connector 56 is connected to the venous connector 41 (FIG. 7); or
the first auxiliary connector 57 is connected to venous connector 41 and the second auxiliary connector 56 is connected to the arterial connector 40 (FIG. 8).

In the recirculation configuration of FIG. 7, full flow rate is present in the blood withdrawal line 6 and in the portion of blood return line 7 that departs from the outlet of the primary chamber 3 and arrives to the non-disconnectable connector which connects the blood return line 7 to the post-dilution fluid line 25; partial flow rate is present in the portions of lines connecting the first auxiliary connector 57 to the second auxiliary connector 56 and then in the portion of the blood return line 7 downstream the post-dilution fluid line 25.

In the third embodiment the first and the second auxiliary connectors are both three-way connectors which are provided each with a disconnectable port 70 configured to be connected to another disconnectable port 72 of one connector selected between the venous connector 40, the arterial connector 41 in accordance to one of the two possible alternatives above identified. The disconnectable port 72 of the first and second auxiliary connectors 57, 56 are of a type which opposite to the type of the disconnectable port of the venous and arterial connectors. In other words, when the disconnectable port 72 of the first and second auxiliary connectors 57, 56 are of a male type, the disconnectable port of the arterial and venous connector is of a female type, and vice-versa.

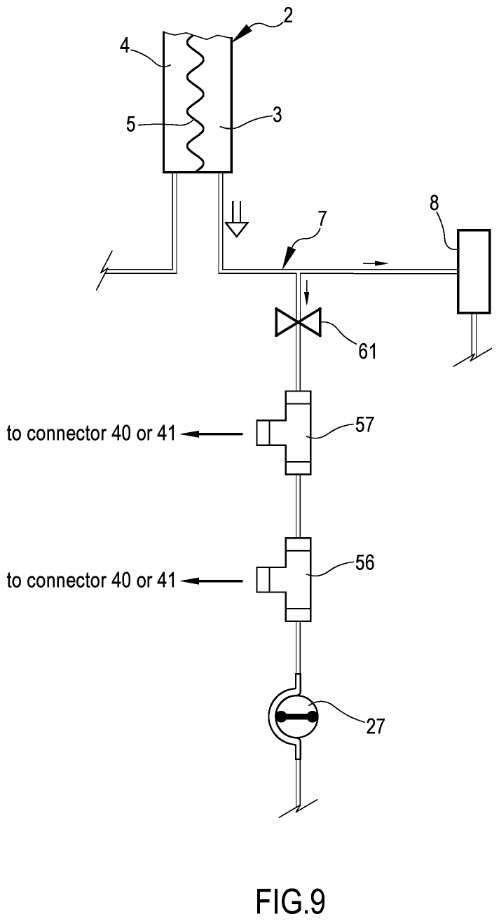

As schematically disclosed in FIG. 9, optionally, the post-dilution fluid line 25 may comprise a pressure drop valve or flow resistor 60, which is in particular installed downstream the first auxiliary connector 57 just upstream the non-disconnectable three way connector that connects the post-dilution fluid line 25 to the blood return line 7. The pressure drop valve or flow resistor 60 is configured to even flow resistances during recirculation and may be detachable or fixedly installed on the fluid line; optionally, the drop valve or flow resistor may be of a variable-resistance type.

### Fourth Embodiment

In a fourth embodiment the first auxiliary connector 59 and the second auxiliary connector 58 are arranged both on the blood circuit, being configured so that the first auxiliary connector 59 is arranged on the blood return line 7 while the second auxiliary connector 58 is arranged on the blood withdrawal line 6. Preferably, albeit in a non-limiting extent, the second auxiliary connector 58 lies on the portion 6a of the blood withdrawal line 6 that once the disposable circuit is properly installed on the apparatus 1, lies upstream the blood pump 11; the first auxiliary connector 59 is preferably installed upstream the bubble detector 8a and the safety clamp 9. Therefore, the first auxiliary connector 59 is arranged downstream the second auxiliary connector 58.

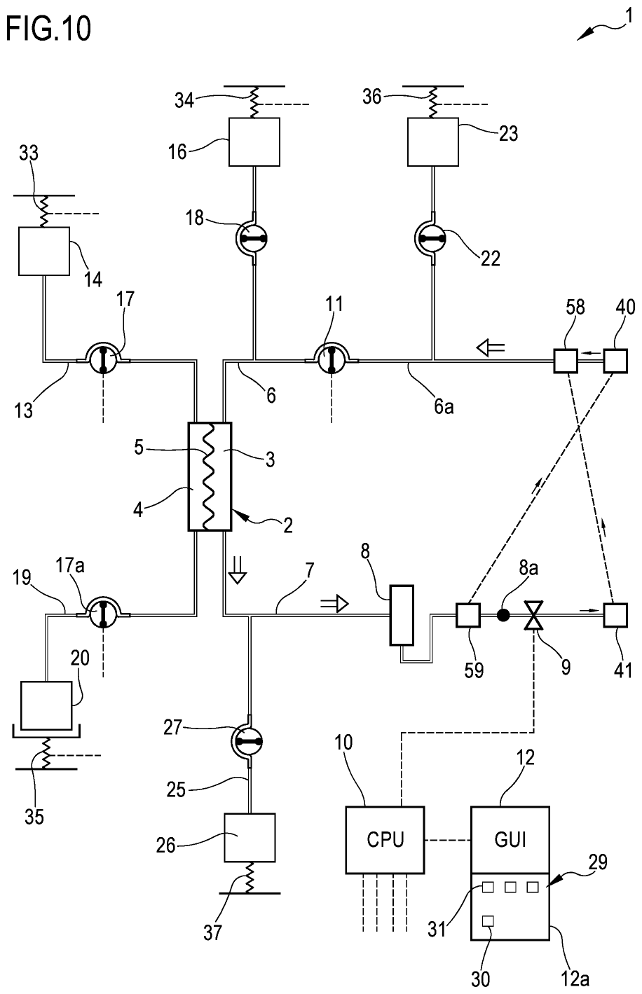

In the recirculation configuration of FIG. 10, the first auxiliary connector 59 is connected to the arterial connector 40 and the second auxiliary connector 58 is connected to the venous connector.

In the recirculation configuration of FIG. 10, full flow rate is present in the blood withdrawal line 6 downstream the second auxiliary connector 58 and upstream the first auxiliary connector 59, i.e. in the respective portions of blood withdrawal lines 6 between the second auxiliary connector 58 and the inlet of the primary chamber 3, and in the portion of blood return line 7 between the outlet of the primary chamber and the first auxiliary connector 59; partial flow rate is present in the portions of blood withdrawal line 6 that lies between the second auxiliary connector 58 and the arterial connector 40 and in the blood return line 7 that lies between the first auxiliary connector 59 and the venous connector 41.

In the fourth embodiment the first and the second auxiliary connectors are both three-way connectors which are provided each with a disconnectable port 70 configured to be connected to another disconnectable port 72 of one connector selected between the venous connector 40, the arterial connector 41. The disconnectable port 72 of the first and second auxiliary connectors 59, 58 are of a type which opposite to the type of the disconnectable port of the venous and arterial connectors. In other words, when the disconnectable port 72 of the first and second auxiliary connectors 59, 58 are of a male type, the disconnectable port of the arterial and venous connector is of a female type, and vice-versa.

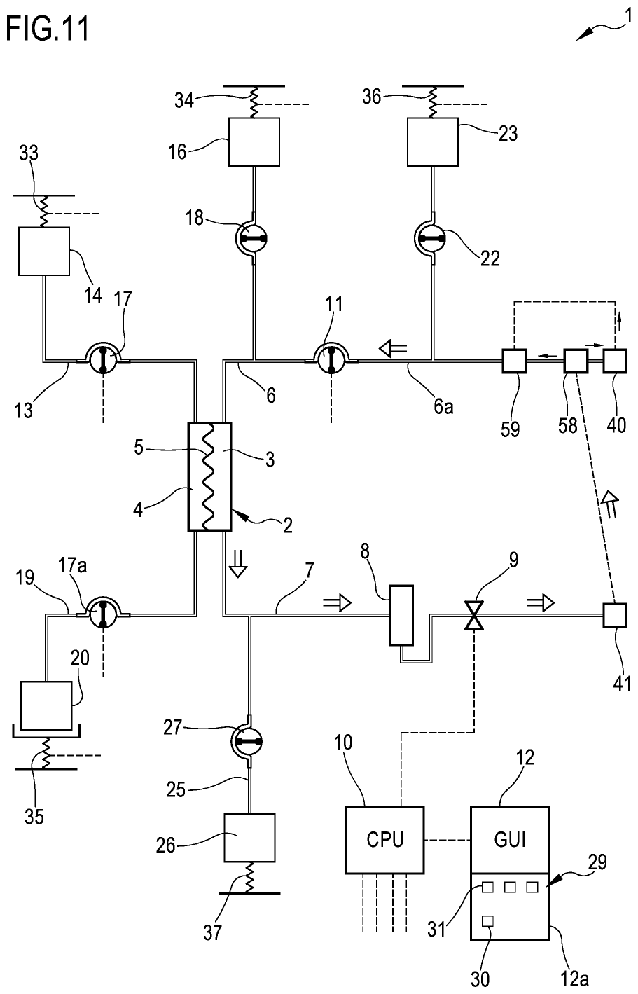

FIG. 11 shows an alternative connection wherein the first and the second auxiliary connectors are both placed on the blood withdrawal line 6, in the line portion 6a upstream the blood pump 11; the venous connector 41 is connected directly to the second auxiliary connector 58; the arterial connector 40 is connected directly to the first auxiliary connector 59 which is placed downstream the second auxiliary connector 58. It may be noted that this latter configuration is not to be considered as preferred but anyway still allowable to define a closed circuit allowing fluid recirculation as intended in the present disclosure.

### Fifth Embodiment

In a fifth embodiment the first auxiliary connector 55 and the second auxiliary connector 54 are arranged both on a single auxiliary line which is installed in a portion of e.g., the blood withdrawal line 6, preferably but in a non-limiting extent, in a portion of the blood withdrawal line that once the disposable circuit is properly arranged on the apparatus 1 lies upstream the blood pump 11. In the fifth embodiment, the first auxiliary connector 55 is arranged downstream the second auxiliary connector 54.

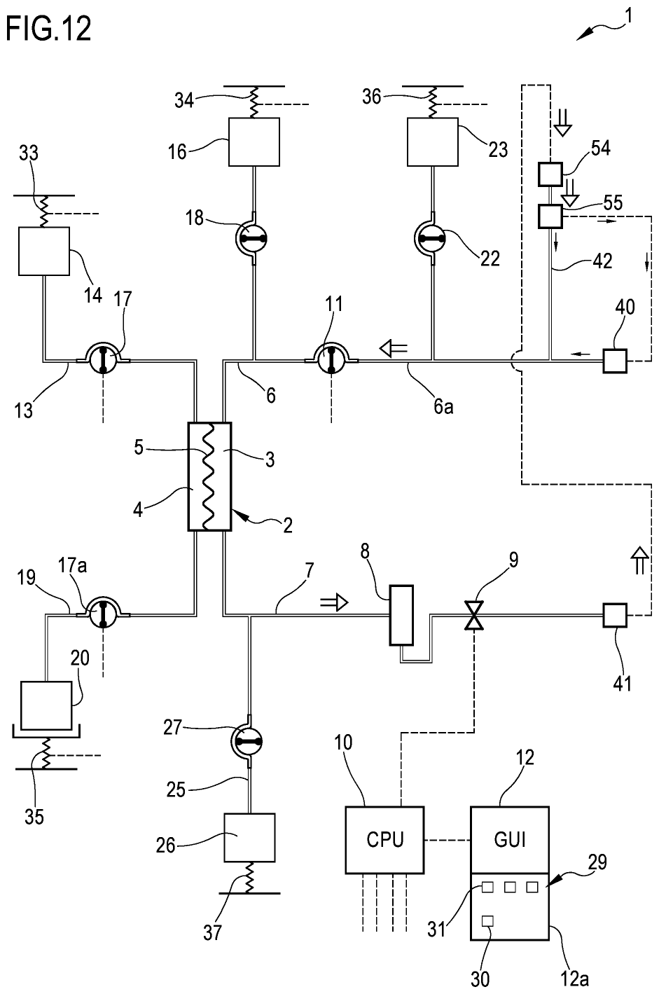

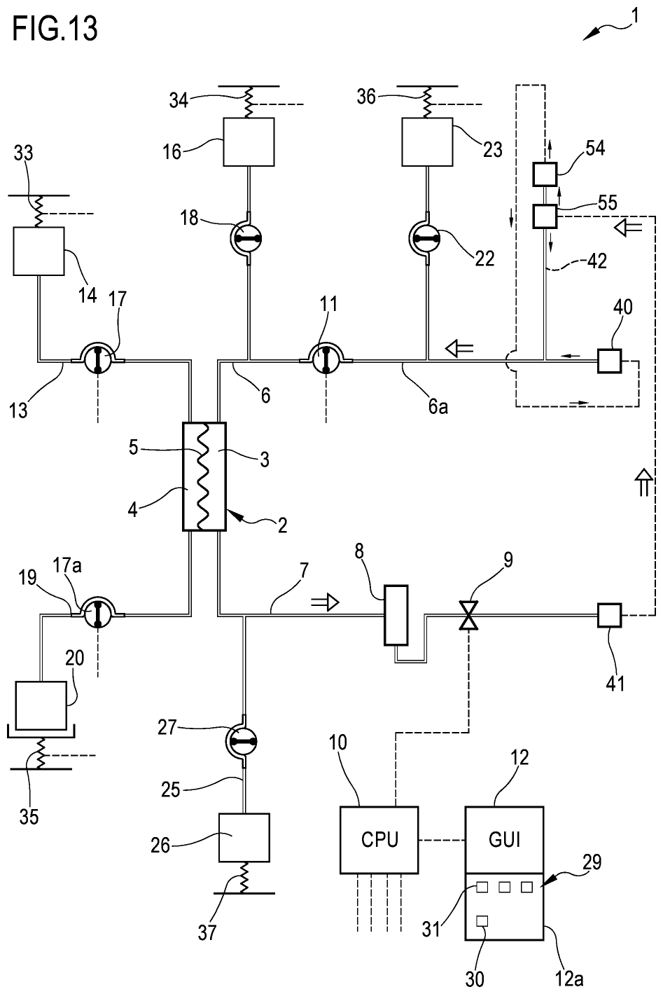

In the recirculation configuration, alternatively:
the first auxiliary connector 55 is connected to the arterial connector 40 and the second auxiliary connector 54 is connected to the venous connector 41 (FIG. 12); or
the first auxiliary connector 55 is connected to venous connector 41 and the second auxiliary connector 56 is connected to the arterial connector 40 (FIG. 13).

In the recirculation configuration of FIG. 12, full flow rate is present in the blood withdrawal line 6 downstream the auxiliary line 42 and in the blood return line 7; partial flow rate is present in the auxiliary line 42 and in the portion of blood withdrawal line 6 which is comprised between the three way non-disconnectable connector which connects one end of the auxiliary line 42 with the blood withdrawal line 6 and the second end of the blood withdrawal line 6 which ends on the arterial connector 40.

In the fifth embodiment one connector between the first and the second auxiliary connectors is a three-way connector which is provided with a disconnectable port 70 configured to be connected to another disconnectable port 72 of one connector selected between the venous connector 40, the arterial connector 41 in accordance to one of the two possible alternatives above identified. The other connector is a two way connector provided with a disconnectable port and a non-disconnectable port fixedly installed on a respective tube portion of the auxiliary line 42. The disconnectable port 72 of the first and second auxiliary connectors 55, 54 are of a type which opposite to the type of the disconnectable port of the venous and arterial connectors. In other words, when the disconnectable port 72 of the first and second auxiliary connectors 55, 54 are of a male type, the disconnectable port of the arterial and venous connector is of a female type, and vice-versa.

The fifth embodiment is similar to the first embodiment; however, a service line 42 instead of a treatment line 21 is used for the temporary disconnection and recirculation.

### Sixth Embodiment

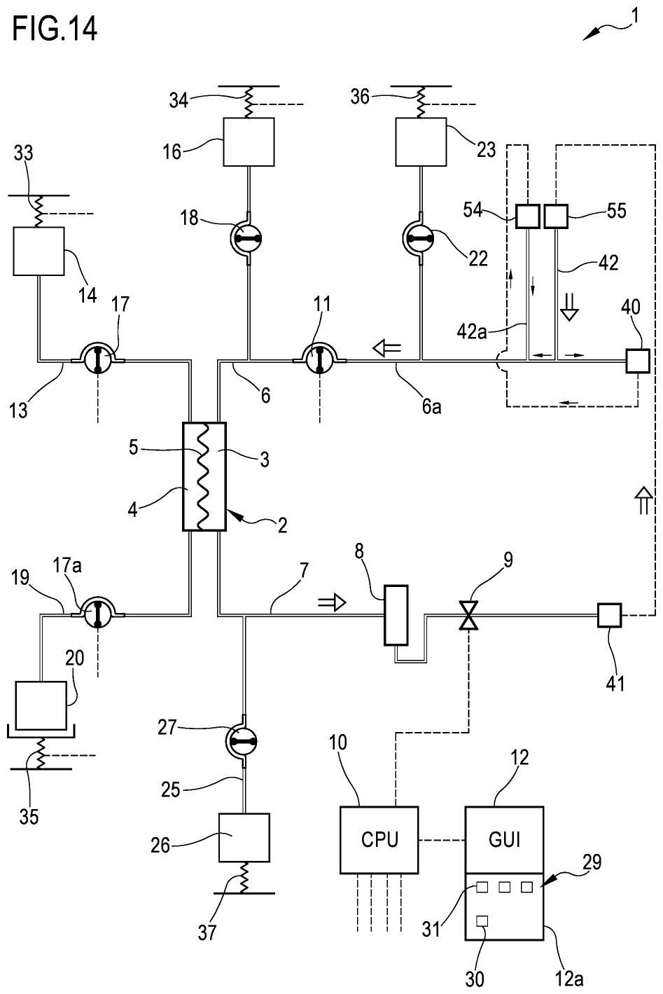

A sixth embodiment of the disposable circuit is schematically shown in FIG. 14; it is provided with a first auxiliary line 42 and a second auxiliary line 42a arranged on a portion of the blood withdrawal line 6 which—preferably albeit in a non-limiting extent—when the disposable circuit is properly arranged on the apparatus 1 lie both upstream the blood pump 11 and optionally upstream the pre-blood pump pre-infusion line 21. Each of the first and second auxiliary lines 42, 42a are provided with a first end connected to the blood withdrawal line 6 and a second end which terminates on a respective first auxiliary connector 55 arranged on the first auxiliary line 42 and a second auxiliary connector 54 arranged on the second auxiliary line 42a.

In the recirculation configuration, alternatively:
the first auxiliary connector 55 is connected to the venous connector 41 and the second auxiliary connector 54 is connected to the arterial connector 40 (FIG. 15); or
the first auxiliary connector 55 is connected to arterial connector 40 and the second auxiliary connector 56 is connected to the venous connector 41 (not depicted in any figure, since substantially symmetrical to the configuration of FIG. 15).

In the recirculation configuration of FIG. 15, full flow rate is present in the blood withdrawal line 6 downstream the second auxiliary line 42a and in the blood return line 7; partial flow rate is present in the first, second auxiliary lines 42, 42a and in the portion of blood withdrawal line 6 which is comprised between the three way non-disconnectable connector which connects one end of the first auxiliary line 42 with the three way non-disconnectable connector which connects one end of the second auxiliary line 42a upstream up to the second end where the blood withdrawal line 6 ends with the arterial connector 40.

In the sixth embodiment both the first and the second auxiliary connectors are two way connectors provided with a disconnectable port and a non-disconnectable port fixedly installed on a respective tube portion of the respective first, second auxiliary line 42. The disconnectable port 72 of the first and second auxiliary connectors 55, 54 are of a type which opposite to the type of the disconnectable port of the venous and arterial connectors. In other words, when the disconnectable port 72 of the first and second auxiliary connectors 55, 54 are of a male type, the disconnectable port of the arterial and venous connector is of a female type, and vice-versa.

Finally, it is not excluded that the first and second auxiliary connectors may be placed one on any of the described infusion lines and the other on the blood circuit, properly allowing for blood recirculation when connected to the arterial and venous connectors 40, 41.

Method of Operation

It is herewith described in detail the method of operation of the disposable circuit according to the present disclosure, in a context wherein a patient may be temporarily disconnected from the apparatus 1, thus also from the disposable circuit, during a blood treatment.

In use, before starting with the treatment configuration, the disposable circuit is installed on the apparatus 1; for easing the installation of the disposable circuit, and as well for reducing the risk of errors performed by the qualified operators during said installation, the apparatus 1 may be provided with visual instructions on the GUI helping the operator in the correct placement. The control unit 10 provides on a screen of the user interface of the apparatus 1 selection items for the machine configuration before the treatment. Once prescription and the relevant parameters are entered, the disposable circuit is properly primed; thereafter, the blood circuit is connected to the patient vascular access and the treatment is started.

In case the treatment has to be temporary interrupted, the user interface has a corresponding screen, wherein a selection item, for instance an icon, for the recirculation configuration may be user selected.

As it is briefly disclosed also in FIG. 24, when the patient has to be disconnected from the apparatus 1, the user selects the recirculation configuration selection item on the screen of the apparatus 1 and then the control unit 10 configures the apparatus 1 in a particular configuration wherein first at least the blood pump 11 and preferably all the pumps of the apparatus 1 are stopped (block 1000). Stopping of at least the blood pump 11 is required for switching or reconfiguring the apparatus 1 to the aforementioned recirculation configuration, and thus the disposable circuit from the treatment configuration to the recirculation configuration and vice-versa, since the blood pump 11 is the pump mainly involved in forcing fluid circulation from the arterial connector to the venous connector in both the operative and in the recirculation configuration.

Albeit in a non-limiting extent, the stopping signal may be sent also to the effluent fluid pump 17 and the dialysis pump 17a. In case dialysis fluid continues circulating in the fluid circuit, at least the ultrafiltration is stopped or set to a minimum very low value. Of course, one or more infusion pumps may run, and the infused fluid be balanced by ultrafiltration.

Then (block 1001) a disconnection of the patient from the apparatus 1 takes place: the user (and/or the apparatus) clamps the blood line ends and then disconnect the arterial connector 40 and venous connector 41 from the access device (e.g., the central catheter or the access needles), so that the arterial connector and the venous connector 40, 41 result both with a respective disconnectable end actually free, ready to be connected to the respective free end of the auxiliary connectors, according to one of the aforementioned embodiments.

Generally, the user disconnects a first connector, normally the arterial connector 40 is firstly disconnected. The disconnected (e.g., arterial) connector is then connected to the first or to the second auxiliary connector, depending on the chosen recirculation configuration. Thereafter, the second connector (e.g., the venous connector 41) is disconnected and subsequently connected to the free connector among the first and the second auxiliary connector defining the recirculation configuration. In other words, the user disconnect and connects the arterial and venous connectors sequentially.

As indicated in block 1002 of FIG. 24, the first auxiliary connector and the second auxiliary connector are then connected to the venous connector 41 and to the arterial connector 40, preferably according to the disclosure of any of the configurations of the embodiments described before. If the first auxiliary connector and the second auxiliary connectors are of a self-sealing type, there is typically no need for the operator to remove closing caps from the disconnectable ports 70, 70a of the auxiliary connectors; otherwise, before properly setting the connection of the auxiliary connectors to the respective arterial connector 40 and venous connector 41, the operator shall properly remove any protective and/or closing cap from the disconnectable port. Of course, in case the self-sealing connector has no cap, there may be a need to clean/disinfect the self-sealing port before making a connection to it.

It may be noted that flexibility of the conduits of the herein disclosed disposable circuit allow for an easy reconfiguration; it may be further noted that during the switching or reconfiguration from the operative configuration to the recirculation configuration, the circuit is left in place on the apparatus 1. In many cases, the portions of the blood circuit 6, 7 closer to both the arterial connector 40 and venous connector 41 may be the ones which are the more easily accessible and/or free or anyway distant from a position at least temporarily defined on pathways on the apparatus 1: therefore, the step of reciprocal connection of an auxiliary connector with either a venous connector 40 or an arterial connector 41 may be preferably performed by moving a portion of the blood circuit 6, 7, so that is physically the venous connector (or the arterial connector) to be brought first closer and then in contact with the free end port of the respective auxiliary connector. This eases the operation for the user, and further reduces the time required for reconfiguring the circuit and the apparatus. Thanks to this aspect the reconfiguration to the recirculation configuration may be rapidly performed.

Once the proper connection between the first auxiliary connector, the second auxiliary connector and the arterial connector 40 and venous connector 41 is set, the blood lines are unclamped and the operator shall promptly intervene on the user interface of the apparatus 1 to set a command of reactivation of at least the blood pump 11. This step is schematically indicated in block 1003 of FIG. 24. Preferably this is made through an interaction with a specific icon provided on a screen of the apparatus 1, or through the action on a specific activation button on the apparatus 1 (block 1003, FIG. 24).

During the recirculation of fluid in the recirculation configuration, it is not wanted that liquids contained in any of the fluid lines connected to the blood circuit 6, 7 are drained therefrom. Thus, according to a recirculation process, any pump of a fluid line connected to the blood circuit 6, 7 is, and rests, deactivated for the entire process of recirculation.

This is particularly suitable when the first auxiliary connector and the second auxiliary connector are arranged both downstream the pumps of the fluid lines, because stopping of the pump acts as a means for impeding free fluid circulation in the respective fluid line upstream the pump. This advantage is further enhanced when the pumps acting on the fluid lines are occlusive pumps, like a peristaltic pump, because this latter type of pump not only provides fluid flow additional resistance when not operative, but substantially fully closes the portion of conduct of the fluid line, physically impeding any draining of fluid from the portion upstream thereof. Hence contamination of the fluids contained upstream the pumps in any of the lines introducing fluids in the blood circuit 6, 7 is prevented or anyway significantly reduced.

In a first alternative, when fluid is recirculated in the disposable circuit in said recirculation configuration, the effluent fluid pump 17 and the dialysis pump 17a are stopped; in another and second alternative, when the fluid is recirculated in the disposable circuit in said recirculation configuration, the effluent fluid pump 17 and the dialysis pump 17a are left active (but no ultrafiltration is achieved).

When the temporary disconnection of the patient ends, i.e. when the patient shall be reconnected to the apparatus 1 through the venous connector 40 and the auxiliary connector 41 for the purposes of finishing the temporarily suspended blood treatment, the user selects a specific command on the user interface operatively connected to control unit 10 which stops the activation of the blood pump 11, and if the effluent fluid pump 17 and the dialysis pump 17a were left active during the recirculation of fluid, also those pumps may be stopped. Then the operator clamps and disconnects the venous connector 40 and arterial connector 41 from the respective first auxiliary connector, second auxiliary connector at which they are connected. The closed configuration for the circuit defined when the recirculation configuration of the disposable circuit was started is then ended, and temporarily the disposable circuit is open. If the first auxiliary connector and the second auxiliary connector are not of a self-sealing type, the operator then provides to close the free disconnectable end thereof; otherwise, if the first auxiliary connector and the second auxiliary connector are of a self-sealing type, this latter action of the operator is not required.

The venous connector 40 and arterial connector 41 are then reconnected to the access device of the patient and unclamped; through the menu of the control unit 10 on the screen, the user selects the selection item corresponding to the restart of the blood treatment, and pumps of the apparatus 1, in particular at least the blood pump 11, is then reactivated. Reactivation is preferably made also for the effluent fluid pump 17 and the dialysis pump 17a, and for all infusion pumps (as required by the set treatment mode) for allowing treatment delivery.

It is noted that the above disconnection procedure does not require to restitute blood to the patient before temporary disconnection (though this is also possible). Therefore, recirculation occurring in the blood lines and eventually in the portion of the infusion line involved occurs to patient blood.

As an alternative procedure, extracorporeal blood may be restituted to the patient before recirculation. According to this embodiment, after the blood pump is stopped and the blood withdrawal line 6 is clamped, the arterial connector 40 is disconnected from the access device of the patient and is connected to a bag containing physiological fluid (e.g., saline or replacement fluid). The blood withdrawal line is unclamped and blood pump reactivated to return blood to the patient via the blood return line which is still connected to the vascular access. Once the blood is restituted, blood withdrawal line is clamped and the arterial connector is properly connected to the first or the second auxiliary connector.

Then the blood return line is clamped, the venous connector 41 removed from the access device and connected to the other one of the first and second auxiliary connector. The user unclamps the blood lines and starts recirculation of the physiological fluid inside the recirculation circuit.

Notably, in case an infusion line is present on the circuit, the content of the bag feeding the infusion line may be used to provide a suitable fluid for restituting blood. Namely, the blood withdrawal line is clamped and the infusion pump (and if necessary the blood pump, too) is activated to force fluid to enter into the blood lines and push blood to the patient via the blood return line and vascular access. This alternative procedure may be performed even before disconnecting the arterial connector.

During recirculation of fluid the usual alarms, such as the access, arterial and venous pressure alarms are deactivated or the alarm windows changed. Indeed, the different conditions during recirculation is not compatible with the usual alarm thresholds and with no change/deactivation would generate several false alarms.

Parts of the process therein described may be provided through a data processing unit, technically interchangeable with one or more data processing units conceived for executing a portion of a software program or a firmware program loaded to a memory support. The software or firmware program may be written in any known programming language. The electronic computers, if two or more, may be connected each other through a data connection such that the computation powers thereof may be shared; the electronic computers may therefore be installed in different positions thereby realizing the data connection in a distributed computing environment. The data processing unit, or control unit, may be a general purpose processor, specifically configured for executing one or more parts of the process defined in the present disclosure through the software or firmware program, or may be an ASIC or a dedicated processor or an FPGA, specifically programmed for executing at least part of the operations of the process herein described. The memory support may be located inside or outside the processor or control unit or data processing unit, and may be geographically remotely located therefrom.

The invention is not limited to the embodiment/s illustrated in the drawings. Accordingly, it should be understood that where features mentioned in the appended claims are followed by reference signs, such signs are included solely for the purpose of enhancing the intelligibility of the claims and are in no way limiting on the scope of the claims.

It is finally clear that any adaptation or addition is made possible on any part of the elements which form part of the present disclosure without for this departing from the scope of protection provided by the annexed claims.

1-29. (canceled)

30. A disposable circuit for extracorporeal treatment of blood comprising:
a filtration unit;
a blood circuit comprising a blood withdrawal line having a first end connected to an inlet of the filtration unit, and a blood return line having a first end connected to an outlet of the filtration unit, the blood withdrawal line and the blood return line configured to connect to a patient cardiovascular system,
wherein the blood withdrawal line has a second end provided with an arterial connector and the blood return line has a second end provided with a venous connector, the arterial connector and the venous connector configured to detachably connect to a vascular access device of a patient;
and wherein the blood circuit is configured to interface with a blood pump to control flow in the blood circuit;
at least one fluid line connected to the blood circuit,
a first auxiliary connector arranged either on the blood circuit or on the at least one fluid line, the first auxiliary connector configured to removably connect with one of the arterial connector and the venous connector in a recirculation configuration to define a closed circuit allowing fluid recirculation in the blood circuit;
a second auxiliary connector arranged on the blood circuit or on the at least one fluid line, wherein the first auxiliary connector and the second auxiliary connector are configured to be removably connected one with the arterial connector and the other with the venous connector to define the closed circuit.

31. A disposable circuit for extracorporeal treatment of blood comprising:
a filtration unit;
a blood circuit comprising a blood withdrawal line having a first end connected to an inlet of the filtration unit, and a blood return line having a first end connected to an outlet of the filtration unit, the blood withdrawal line and the blood return line configured to connect to a patient cardiovascular system,
wherein the blood withdrawal line has a second end provided with an arterial connector and the blood return line has a second end provided with a venous connector, the arterial connector and the venous connector configured to detachably connect to a vascular access device of a patient; and wherein the blood circuit is configured to interface with a blood pump to control flow in the blood circuit;
a first auxiliary connector arranged on the blood circuit, the first auxiliary connector configured to removably connect with one of the arterial connector and the venous connector in a recirculation configuration to define a closed circuit allowing fluid recirculation in the blood circuit;
a second auxiliary connector arranged on the blood circuit, and wherein the first auxiliary connector and the second auxiliary connector are configured to be removably connected one with the arterial connector and the other with the venous connector to define the closed circuit.

32. The disposable circuit of claim 30, wherein the disposable circuit is reversibly configurable between a treatment configuration in which the arterial connector and the venous connector are removably connected to the vascular access device of a patient and blood is withdrawn from the patient through the blood withdrawal line, circulated through the filtration unit and returned to the patient through the blood return line; and
a recirculation configuration in which the arterial connector and the venous connector are removably connected to the first auxiliary connector and the second auxiliary connector, wherein in the recirculation configuration a fluid contained in the blood circuit is recirculated inside the disposable circuit.

33. The disposable circuit of claim 30, wherein both the first auxiliary connector and the second auxiliary connector comprise three ports including a disconnectable port configured to be connected to one connector selected among the venous connector and the arterial connector;
wherein both the first auxiliary connector and the second auxiliary connector are provided with:
a first and a second non-disconnectable service port fixed to respective tube portions;
the disconnectable port.

34. The disposable circuit of claim 30, wherein the at least one fluid line comprises one or more of the following lines:
a post-blood pump pre-dilution infusion line directly connected to the blood withdrawal line;
a pre-blood pump pre-dilution infusion line directly connected to the blood withdrawal line;
a post-dilution fluid line directly connected to the blood return line;
wherein the first auxiliary connector comprises a first and a second non-disconnectable service port fixed to a respective tube portion of the fluid line at which the first auxiliary connector is arranged.

35. The disposable circuit of claim 30, wherein the second auxiliary connector comprises a first and a second non-disconnectable service port fixed to a respective tube portion of the fluid line at which the second auxiliary connector is arranged, the first auxiliary connector and the second auxiliary connector being fixed to the same fluid line.

36. The disposable circuit of claim 30, comprising a pre-blood pump pre-dilution infusion line directly connected to the blood withdrawal line and wherein the first auxiliary connector and the second auxiliary connector are fixed to the pre-blood pump pre-dilution infusion line, wherein in the recirculation configuration the first auxiliary connector fixed on the pre-blood pump pre-dilution infusion line is connected to the arterial connector and the second auxiliary connector fixed on the pre-blood pump pre-dilution infusion line is connected to the venous connector.

37. The disposable circuit of claim 36, wherein the first auxiliary connector is arranged on the pre-blood pump pre-dilution infusion line downstream the second auxiliary connector.

38. The disposable circuit of claim 30, comprising a pre-blood pump pre-dilution infusion line configured to allow fluid administration to the blood circuit downstream the arterial connector; the pre-blood pump pre-dilution infusion line comprising a peristaltic pump tract configured to be coupled with a corresponding peristaltic pump and a container, the pre-blood pump pre-dilution infusion line being connected to the container, the first auxiliary connector and the second auxiliary connector being connected to the pre-blood pump pre-dilution infusion line downstream from the peristaltic pump tract.

39. The disposable circuit of claim 30, comprising a first pressure drop valve or flow resistor configured to even fluid flow distribution during recirculation, the first pressure drop valve or flow resistor being arranged on the blood circuit or on the at least one fluid line, the first pressure drop valve or flow resistor configured to even respective fluid flow resistances of two connected branch tube portions during recirculation, wherein the first pressure drop valve or flow resistor is arranged in one of the two connected branch tube portions comprising a portion of the fluid line and a portion of the blood circuit.

40. The disposable circuit of claim 30, further comprising:
an effluent fluid line connected to an outlet of the secondary chamber, and
a dialysis fluid line connected to the inlet of the secondary chamber,
the disposable circuit being a continuous renal replacement therapy (CRRT) disposable circuit.

41. The disposable circuit of claim 30, wherein the first auxiliary connector and the second auxiliary connector are arranged on the blood circuit, wherein in the recirculation configuration, the first auxiliary connector arranged on the blood return line is connected to the arterial connector and the second auxiliary connector arranged on the blood withdrawal line is connected to the venous connector.

42. The disposable circuit of claim 30, wherein both the first auxiliary connector and the second auxiliary connector comprise a disconnectable port configured to be connected to a corresponding disconnectable port of either the venous connector or the arterial connector.

43. The disposable circuit of claim 42, wherein both the first auxiliary connector and the second auxiliary connector comprise three ports including a disconnectable port configured to be connected to a disconnectable port the venous connector or the arterial connector.

44. The disposable circuit of claim 43, wherein the disconnectable port of the first auxiliary connector and the disconnectable port of the arterial connector or of the venous connector are either a first-male-type or a second-female-type, the first-male-type being configured to introduce into the second-female-type;
both the arterial connector and the venous connector are connectors provided with a disconnectable port of the first-male-type, or
both the arterial connector and the venous connector are provided with a disconnectable port of the second-female-type.

45. The disposable circuit of claim 44, wherein the first auxiliary connector and the second auxiliary connector comprise the detachable port both of the first-male-type or both of the second-female-type.

46. The disposable circuit of claim 30, wherein the first auxiliary connector and the second auxiliary connector are installed on the infusion line, one of the first auxiliary connector and the second auxiliary connector upstream with respect to the other on the infusion line, the infusion line comprising a fluid pump tract on which a fluid pump operates, the first auxiliary connector and the second auxiliary connector being installed on the infusion line downstream the fluid pump tract.

47. The disposable circuit of claim 30, being a continuous renal replacement therapy (CRRT) disposable circuit configured for use in a renal failure apparatus for extracorporeal treatment of blood in the acute field, the filtration unit comprising a primary chamber connected to the blood circuit using the inlet and the outlet and a secondary chamber separated from the primary chamber by a semi-permeable membrane, the continuous renal replacement therapy (CRRT) disposable circuit comprising a disposable dialysis fluid line irremovably connected to an inlet of the secondary chamber of the filtration unit and a disposable effluent line irremovably connected to an outlet of the secondary chamber of the filtration unit.

48. The disposable circuit of claim 30, wherein in the recirculation configuration fluid flows from the arterial connector through the filtration unit to the venous connector, from the venous connector to the second auxiliary connector and from the second auxiliary connector to the first auxiliary connector and from the first auxiliary connector to the arterial connector.

49. The disposable circuit of claim 30, wherein the first auxiliary connector and the second auxiliary connector are separate connectors.

50. The disposable circuit of claim 30 mounted on an apparatus for extracorporeal treatment of blood, the apparatus comprising a control unit and a blood pump configured to control flow of blood in the blood circuit,
wherein the control unit is configured to:
stop the blood pump;
receive a command of activation of a fluid recirculation in the blood circuit;
when the disposable circuit is in a recirculation configuration, activate the blood pump, to cause recirculation of fluid inside the blood circuit.

51. The disposable circuit of claim 50, wherein in the recirculation configuration, the first auxiliary connector is directly connected either with the arterial connector or with the venous connector and the second auxiliary connector is directly connected with the other one of the arterial connector and the venous connector, and wherein the control unit is configured to maintain the disposable circuit in the recirculation configuration wherein during recirculation either a full flow rate of fluid entering into the first auxiliary connector or into the second auxiliary connector is split into separate partial flow rates or two partial flow rates of fluid entering into the first auxiliary connector or into the second auxiliary connector are merged and exit as a full flow rate.

52. The disposable circuit of claim 50, wherein, in the recirculation configuration, the first auxiliary connector is directly connected either with the arterial connector or with the venous connector and the second auxiliary connector is directly connected with the other one of the arterial connector and the venous connector, and wherein the control unit is configured to maintain the disposable circuit in the recirculation configuration wherein during recirculation either a full flow rate of fluid entering into the first auxiliary connector or into the second auxiliary connector is split into separate partial flow rates or two partial flow rates of fluid entering into the first auxiliary connector or into the second auxiliary connector are merged and exit as a full flow rate.

53. The disposable circuit of claim 50, wherein, in a treatment configuration, the control unit is configured to:
receive or calculate initial values to one or more fluid flow rates selected in the group including a fluid flow rate through an effluent line, a fluid flow rate through the at least one fluid line, a fluid flow rate through a dialysis liquid fluid line, a fluid removal rate from the patient, and a blood flow rate; and
actuate one or more of the blood pump, an infusion pump, a dialysis pump, and an effluent liquid pump to deliver the prescribed or calculated flow rate.

54. The disposable circuit of claim 50, wherein, to activate the recirculation configuration, the control unit is configured to:
receive a command of activation of a temporary fluid recirculation in the blood circuit;
set a value of recirculation flow rate through the blood circuit;
activate the blood pump to cause recirculation of fluid in the blood circuit when in the recirculation configuration;
wherein the recirculation configuration takes place after the treatment configuration is interrupted and the flow rate of fluid in the blood circuit is reduced to zero by deactivating the blood pump and one or more infusion pumps.

55. The disposable circuit of claim 54, wherein the control unit is configured to receive an electronic confirmation of disconnection of the arterial connector and venous connector before activating the blood pump for recirculation.

56. The disposable circuit of claim 50, wherein, in a treatment configuration, the first auxiliary connector and the second auxiliary connector are neither connected to the arterial connector nor to the venous connector, and wherein, in the recirculation configuration, the first auxiliary connector is directly connected with one connector between the arterial connector and the venous connector and the second auxiliary connector is directly connected with the other of the arterial connector and the venous connector.
